(19) United States
(12) Patent Application Publication
(10) Pub. No.: US 2011/0177538 A1
(43) Pub. Date: Jul. 21, 2011

(54) BIOMARKERS FOR RHEUMATOID ARTHRITIS (RA)

(76) Inventors: Sara Mangialaio, Buschwiller (FR); Teresa Urbanowska, Basel (CH); Peter Grass, Schopfheim (DE)

(21) Appl. No.: 11/572,559

(22) PCT Filed: Jul. 22, 2005
(86) PCT No.: PCT/EP2005/008042

§ 371 Date: Mar. 6, 2008

Related U.S. Application Data
(60) Provisional application No. 60/590,635, filed on Jul. 23, 2004.

(51) Int. Cl. G01N 33/53; G01N 33/68; C12Q 1/02

(52) U.S. Cl. 435/7.94; 436/86; 435/29; 436/501; 435/7.92

(57) ABSTRACT

The invention provides methods for predicting and compositions for use in the prediction of rheumatoid arthritis (RA) based upon the simultaneous measurement of at least two biomarkers in a biological sample. The invention further relates to methods for following the efficiency of a treatment against RA and to methods for identifying test substances that are likely to prevent or diminish RA.

### BACKGROUND OF THE INVENTION

Rheumatoid arthritis (RA) is an inflammatory, autoimmune, systemic disease of unknown pathogenesis. Rheumatoid arthritis affects about 1% of the Caucasian population in a female to male ratio of 2.5/1 (Lee & Weinblatt 2001; Lancet, vol. 358, no. 9285, pp. 903-911). The disease can occur at any age, but it is most common among those aged between 30 to 55 years (Sweeney & Firestein 2004; Int. J. Biochem. Cell Biol., vol. 36, no. 3, pp. 372-378), with its incidence increasing with age.

The predominant symptoms of RA are pain, stiffness, and swelling of peripheral joints. The clinical manifestation of the disorder is very variable, ranging from mild, self-limiting arthritis to rapidly progressive multi-system inflammation with profound morbidity and mortality (Lee & Weinblatt 2001; ibid; Sweeney & Firestein 2004; ibid). Joint damage occurs early in the course of rheumatoid arthritis; 30 percent of patients have radiographic evidence of bony erosions at the time of diagnosis, and this proportion increases to 60 percent by two years (van der Heijde 1985; Br. J. Rheumatol., vol. 34 Suppl 2, pp. 74-78).

RA is a polyarthritis, that involves many joints (six or more), although in the early stages of the disease, only one or a few joints might be afflicted. Virtually all peripheral joints can be affected by the disease; however, the most commonly involved joints are those of the hands, feet and knees (Smolen et al. 1995; Arthritis Rheum., vol. 38, no. 1, pp. 38-43). In addition, RA can affect the spine, and atlanto-axial joint involvement is common in longer-standing disease, and constitutes a directly joint-related cause of mortality. Extra-articular involvement is another hallmark of RA, and this can range from rheumatoid nodules to life-threatening vasculitis (Smolen & Steiner 2003; Nat. Rev. Drug Discov., vol. 2, no. 6, pp. 473-488).

RA is a disease in which the immune and inflammatory systems are closely linked to the destruction of cartilage and bone. Although the link of the two systems remains elusive, and the cause of RA unknown, many pathways involved in the generation of the disease have been recognized. Research over the last few years identified that genetic and infectious factors are involved in RA pathogenesis in a complex interrelated way (Smith & Haynes 2002; Ann. Intern. Med., vol. 136, no. 12, pp. 908-922). Soluble cytokines and chemokines have been shown to be associated with rheumatoid arthritis (Szekanecz & Koch 2001; Curr. Rheumatol. Rep., vol. 3, no. 1, pp. 53-63) such as IL-1β, TNFα and IL-1ra (Gabay et al. 1997; J. Rheumatol., vol. 24, no. 2, pp. 303-308), IL-6 (Arvidson et al. 1994; Ann. Rheum. Dis., vol. 53, no. 8, pp. 521-524), IL-8 and MCP-1 (De Benedetti et al. 1999; J. Rheumatol., vol. 26, no. 2, pp. 425-431), and serum amyloid A (SAA) that is related to an inflammation process.

Drugs constitute the main form of treatment for RA. The currently used drugs are divided in four major categories: a) nonsteroidal anti-inflammatory drugs (NSAIDs); b) disease-modifying anti-rheumatic drugs (DMARDs), c) steroids and d) analgesics.

Historically, the treatment of RA had been based on the use of NSAID drugs (such as ibuprofen and aspirin) to reduce swelling and pain associated with the disease. An approach which does not, however, tackle the underlying cause(s) of the pain associated with the disease. This approach has been superseded with the development of disease-modifying anti-rheumatic drugs (DMARDs) such as sulfasalazine and methotrexate. Another type of DMARDs include biological agents. Approved biological DMARDs include for example Infliximab, Etanercept, Adalimumab or Anakinra.

The diagnosis of RA is based on the use of seven diagnostic criteria recognized by The American Rheumatism Association (ARA) (Arnett et al. 1988; Arthritis Rheum., vol. 31, no. 3, pp. 315-324). The diagnosis requires clinical factors and, therefore, depend on the clinician's asking insightful questions and recognizing the often-subtle early physical findings. The ARA criteria include:
1) morning stiffness in and around joints lasting at least 1 hour before maximal improvement;
2) soft tissue swelling (arthritis) of 3 or more joint areas observed by a physician;
3) swelling (arthritis) of the hand joints
4) symmetric swelling (arthritis);
5) rheumatoid nodules;
6) elevated levels of serum rheumatoid factor (RF);
7) radiographic changes in hand and/or wrist joints.

The first four criteria must be present for a minimum of six weeks before a diagnosis of rheumatoid arthritis can be made. The RA test measures rheumatoid factor—the IgM autoantibody reactive with the epitopes in the Fc region of the IgG molecule (Corper et al. 1997; Nat. Struct. Biol., vol. 4, no. 5, pp. 374-381). Although RF is primarily associated with RA, these antibodies have low disease specificity and can be detected in sera from normal elderly people, healthy individuals, and patients with other autoimmune disorders or chronic infections (Williams D G 1998).

Thus, there exists a need for alternative methods of diagnosing RA, and for methods for following the efficiency of a treatment against RA.

### SUMMARY OF THE INVENTION

In one aspect the invention provides a method for determining whether a subject has or is likely to develop rheumatoid arthritis (RA), said method comprising determining the amounts of at least two biomarkers selected from the group consisting of tumor necrosis factor-alpha (TNF-alpha, TNFα, TNF-a); macrophage chemoattractant protein-1 (MCP-1), IL-1 receptor antagonist (IL-1Ra; IL-1ra), interleukin-8 (IL-8), interleukin-6 (IL-6) and interleukin-1beta (IL-1beta, IL-1β, IL-1b) in a biological sample obtained from said subject.

A further aspect of the invention relates to a method for identifying a test substance that is likely to prevent or diminish RA in a subject, said method comprising the steps of a) determining the amounts of at least two biomarkers selected from the group consisting of TNF-alpha, MCP-1, IL-1ra, IL-8, IL-6 and IL-1 beta in a biological sample obtained from said subject; b) contacting the biological sample with a test substance; and c) determining again the amounts of the at least two biomarkers selected from the group consisting of TNF-alpha, MCP-1, IL-1ra, IL-8, IL-6 and IL-1beta in the biological sample of step b; wherein an alteration in the amounts of the at least two biomarkers in the biological sample of step b) when compared to the amounts of the at least two biomarkers of step a) identifies a test substance that is likely to prevent or diminish RA.

Another aspect of the invention provides a method for following the efficiency of a treatment against RA said method comprising comparing the amounts of at least two biomarkers selected from the group consisting of TNF-alpha, MCP-1, IL-1ra, IL-8, IL-6 and IL-1beta in a biological sample obtained from a subject suffering from RA before initiating said treatment with the amounts of said at least two biomarkers in a biological sample obtained from said subject after initiating said treatment, wherein similar amounts in both biological samples indicates that the treatment is not effective, and wherein an alteration in the amounts of the at least two biomarkers in the biological sample obtained from said subject after initiating said treatment indicates that the treatment is effective.

A still further aspect of the invention relates to a composition for the prediction, diagnosis or prognosis of rheumatoid arthritis (RA) comprising at least two detection agents for determining the amounts of at least two biomarkers selected from the group consisting of TNF-alpha, MCP-1, IL-1ra, IL-8, IL-6 and IL-1beta.

The invention also provides a diagnostic or drug discovery kit, comprising a composition according to the invention and instructions for use. And another aspect of the invention relates to the use of such diagnostic or drug discovery kit in one of the methods of the invention.

### BRIEF DESCRIPTION OF THE FIGURES

FIG. 1: Shows the result of the addition of glycerol into a coating solution.

FIG. 2: Coating antibody titration. Different concentration of coating IL-1β, IL-6 and IL-8 antibody was printed (from left to right Bkg, 25 μg/mL, 50 μg/mL, 100 μg/mL, 200 μg/mL). The best signal-to-noise ratio was measured for the concentration of 200 μg/mL of coating antibody for all analytes.

FIG. 3: Assay was performed using different concentrations of biotinylated antibody (Ab; from left to right: 62.5 ng/mL, 125 ng/mL, 250 ng/mL, 500 ng/mL of bio ab). The best signal-to-noise ratio was generated with the concentration of 500 ng/mL of biotinylated Ab for each analyte. The signal-to noise ratios shown were generated for IL-8 assay.

FIG. 4: Representative IL-6 standard curve for an assay performed with two step and co-incubation protocol. Circle represents IL-6 1-step protocol and rectangle represents IL-6 2-step protocol. The obtained results show that 1 step protocol can improve assay sensitivity.

FIG. 5: Images generated with a non-parallax tray device. Three different image modes are compared: high (left), standard (middle) and high resolution mode (right).

FIG. 6: Correlation of antibody array and ELISA procedures. Cytokines in 78 sera and 10 spiked samples were quantified in parallel using either antibody microarray or ELISA. Data from these two analyses were plotted against each other and the correlation coefficients determined by linear regression analysis.

### DETAILED DESCRIPTION OF THE INVENTION

All patent applications, patents and literature references cited herein are hereby incorporated by reference in their entirety.

In practicing the present invention, many conventional techniques in molecular biology, microbiology, and recombinant DNA are used. These techniques are well known and are explained in, for example. Current Protocols in Molecular Biology, Volumes I, II, and III, 1997 (F. M. Ausubel ed.); Sambrook et al., 1989, Molecular Cloning; A Laboratory Manual, Second Edition, Cold Spring Harbor Laboratory Press, Cold Spring Harbor, N.Y.; DNA Cloning; A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The invention is based at least in part on the discovery of biomarker profiles of subjects having RA. Preferably, the invention relates to the determination of a RA biomarker profile by determining the amount of at least two biomarkers selected from the group consisting of TNF-alpha, MCP-1, IL-1ra, IL-8, IL-6, IL-1beta and RF in a biological sample, most preferably by the use of a composition such as a protein microarray according to the invention.

The biological sample may be a blood sample or tissue sample. Suitable samples include whole blood, serum, semen, saliva, tears, urine, fecal material, sweat, buccal smears, skin, and biopsies of specific organ tissues, such as muscle or nerve tissue and hair. Most preferably, the biological sample is serum.

The subject from whom the biological sample is obtained may be treated with a disease-modifying anti-rheumatic drug (DMARD) such as sulfasalazine, methotrexate, Infliximab, Etanercept, Adalimumab or Anakinra, or Cyclosporin.

A biomarker profile determination according to the methods of the invention may comprise the measurement of nucleic acids or of proteins in a subject or in a biological sample isolated from said subject. In one preferred embodiment of the invention the biomarker determination comprises determination of the amount of mRNA encoding a biomarker. RNA including also mRNA can be isolated from the samples by methods well known to those skilled in the art as described, e.g., in Ausubel et al., Current Protocols in Molecular Biology, Vol. 1, pp. 4.1.1-4.2.9 and 4.5.1-4.5.3, John Wiley & Sons, Inc. (1996). Methods for detecting the amount of mRNA are well known in the art and include, but are not limited to, northern blotting, reverse transcription PCR, real time quantitative PCR and other hybridization methods. The amount of mRNA is preferably determined by contacting the mRNAs with at least one sequence-specific oligonucleotide. In a preferred embodiment said mRNA is determined with two sequence-specific oligonucleotides. The sequence-specific oligonucleotides are preferably of sufficient length to specifically hybridize only to the mRNA encoding the biomarker or to a cDNA prepared from said mRNA. As used herein, the term “oligonucleotide” refers to a single-stranded nucleic acid. Generally the sequence-specific oligonucleotides will be at least 15 to 20 nucleotides in length, although in some cases longer probes of at least 20 to 25 nucleotides will be desirable. The sequence-specific oligonucleotide can be labeled with one or more labeling moieties to permit detection of the hybridized probe/target polynucleotide complexes. Labeling moieties can include compositions that can be detected by spectroscopic, biochemical, photochemical, bioelectronic, immunochemical, and electrical optical or chemical means. Examples of labeling moieties include, but are not limited to, radioisotopes, e.g., ³²P, ³³P, ³⁵S, chemiluminescent compounds, labeled binding proteins, heavy metal atoms, spectroscopic markers such as fluorescent markers and dyes, linked enzymes, mass spectrometry tags, and magnetic labels. Oligonucleotide arrays for mRNA or expression monitoring can be prepared and used according to techniques which are well known to those skilled in the art as described, e.g., in Lockhart et al., Nature Biotechnology, Vol. 14, pp. 1875-1680 (1996); McGall et al., Proc. Natl. Acad. Sci. USA, Vol. 93, pp. 13555-13460 (1996); and U.S. Pat. No. 6,040,138.

A particularly useful method for determining the amount of mRNA encoding the at least two biomarkers of the invention involves hybridization of labeled mRNA to an ordered array of sequence-specific oligonucleotides. Such a method allows the simultaneously determination of the mRNA amounts of the at least 2, at least 3 or 4, or at least 5 or 6 biomarkers selected from the group consisting of TNF-alpha, MCP-1, IL-1ra, IL-8, IL-6 and IL-1beta, and RF. The sequence-specific oligonucleotides utilized in this hybridization method typically are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc.

According to another preferred embodiment of the invention the determining the amounts of at least two biomarkers selected from the group consisting of TNF-alpha, MCP-1, IL-1ra, IL-8, IL-6 and IL-1beta is performed by measuring the amount of protein, i.e. the amount of TNF-alpha, MCP-1, IL-1ra, IL-8, IL-6 and/or IL-1beta protein. In another preferred embodiment of the invention the amount of RF is determined in addition to the amounts of said at least two biomarkers.

The term “protein” as used herein may be used synonymously with the term “polypeptide” or may refer to, in addition, a complex of two or more polypeptides which may be linked by bonds other than peptide bonds, for example, such polypeptides making up the protein may be linked by disulfide bonds. Most preferably, said biomarkers according to the invention comprise the TNF-alpha, MCP-1, IL-1ra, IL-8, IL-6 and/or IL-1beta proteins, and/or RF. The term “protein” may also comprehend a family of polypeptides having identical amino acid sequences but different post-translational modifications, particularly as may be added when such proteins are expressed in eucaryotic hosts. These proteins can be either in their native form or they may be immunologically detectable fragments of the proteins resulting, for example, from proteolytic breakdown. By “immunologically detectable” is meant that the protein fragments contain an, epitope which is specifically recognized by e.g. mass spectrometry or antibody reagents as described below.

In a preferred embodiment the amounts of said at least two biomarkers or biomarker proteins are determined by means of mass spectrometry. According to a more preferred embodiment of the invention the amounts of the at least two biomarkers are determined by means of reagents which specifically bind to the at least two biomarker proteins, wherein the proteins are selected from the group consisting of TNF-alpha, MCP-1, IL-1ra, IL-8, IL-6 and IL-1beta, and RF. In an embodiment of the invention the reagents which specifically bind to the at least two biomarkers comprise aptamers, short DNA or RNA molecules originating from in vitro selection approaches referred to as SELEX (systematic evolution of ligands by exponential enrichment) and having with high affinities to specific ligands, including proteins (Brady et al. 1999; Mol. Diagn., vol. 4, no. 4, pp. 381-388). More preferably, the reagent which specifically binds to the biomarker comprises an antibody (Ab), an antibody derivative and an antibody fragment. Most preferably the reagent is a monoclonal antibody (mAb). Most preferably said antibodies are those described in the Examples further below.

As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein or a fragment of the protein. Such antibodies may be of any immunoglobulin class, including IgG, IgM, IgE, IgA, IgD, and any subclass thereof. A hybridoma producing the mAb of this invention may be cultivated in vitro or in vivo. A chimeric antibody is a molecule in which different portions are derived from different animal species, such as those having a variable or hypervariable region derived from a murine mAb and a human immunoglobulin constant region. Alternatively, the antibody may comprise a single-chain antibody. Techniques described for the production of single-chain antibodies (U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,593,761; 5,585,089; 5,530,101; 5,569,825; 5,625,126; 5,633,425; 5,789,650; 5,661,016; and 5,770,429. Antibody fragments which recognize specific epitopes of the biomarkers of the invention may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science, Vol. 246, pp. 1275-1281 (1989)) to allow rapid and easy identification of monoclonal Fab fragments with the desired specificity. The amount of the biomarker/biomarker proteins of the invention may be determined by immunoassay methods utilizing the antibodies described above. Such immunoassay methods include, but are not limited to, direct or indirect immunoassay such as for example a competitive binding assay, a non-competitive binding assay, a radioimmunoassay, immunohistochemistry, an enzyme-linked immunosorbent assay (ELISA), a sandwich assay, a gel diffusion immunodiffusion assay, an agglutination assay, dot blotting, a fluorescent immunoassay such as fluorescence-activated cell sorting (FACS), chemiluminescence immunoassay, immunoPCR immunoassay, a protein A or protein G immunoassay, and an immunoelectrophoresis assay such as western blotting and others commonly used and widely described in scientific and patent literature, and many employed commercially.

In the case of an enzyme immunoassay, an enzyme is conjugated to the second antibody, usually by means of glutaraldehyde or periodafe. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of secreted protein or fragment thereof. Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed.

In a preferred embodiment of the invention said reagents which specifically bind to the at least two biomarker proteins are immobilized on a solid support such as for example a polystyrene surface. A preferred embodiment of the invention provides a protein microarray (Templin et al. 2004; Comb. Chem. High Throughput. Screen., vol. 7, no. 3, pp. 223-229) for simultaneous binding and quantification of the at least two biomarker proteins according to the invention. The protein microarray consists of molecules (capture agents) bound to a defined spot position on a support material. The array is then exposed to a complex protein sample. Capture agents such as antibodies are able to bind the protein of interest from the biological sample. The binding of the specific analyte proteins to the individual spots can then be monitored by quantifying the signal generated by each spot (MacBeath 2002; Nat. Genet., vol. 32 Suppl, pp. 526-532; Zhu & Snyder 2003; Curr. Opin. Chem. Biol., vol. 7, no. 1, pp. 55-63). Protein microarrays can be classified into two major categories according to their applications. These are defined as protein expression microarrays, and protein function microarrays (Kodadek 2001; Chem. Biol., vol. 8, no. 2, pp. 105-115). Protein expression microarrays mainly serve as an analytic tool, and can be used to detect and quantify proteins, antigen or antibodies in a biological fluid or sample. Protein function microarrays on the other hand can be used to study protein-protein, enzyme-substrate and small molecule-protein interactions (Huang 2003; Front Biosci., vol. 8, p. d559-d576). Protein microarrays also come in many structural forms. These include two-dimensional microarrays constructed on a planar surface, and three-dimensional microarrays which use a ‘flow-through’ support.

Types of protein microarray set-ups: reverse phase arrays (RPAs) and forward phase arrays (FPAs) (Liotta et al. 2003; Cancer Cell, vol. 3, no. 4, pp. 317-325). In RPAs a small amount of a tissue or cell sample is immobilized on each array spot, such that an array is composed of different patient samples or cellular lysates. In the RPA format, each array is incubated with one detection protein (e.g., antibody), and a single analyte endpoint is measured and directly compared across multiple samples. In FPAs, capture agents, usually an antibody or antigen, are immobilized onto the surface and act as a capture molecule. Each spot contains one type of immobilized antibody or capture protein. Each array is incubated with one test sample, and multiple analytes are measured at once.

One of the most common forms of FPAs is an antibody microarray. Antibody microarrays can be produced in two forms, either by a sandwich assay or by direct labeling approach. The sandwich assay approach utilizes two different antibodies that recognize two different epitopes on the target protein. One antibody is immobilized on a solid support and captures its target molecule from the biological sample. Using the appropriate detection system, the labelled second antibody detects the bound targets. The main advantage of the sandwich assay is its high specificity and sensitivity (Templin, Stoll, Bachmann, & Joos 2004; Comb. Chem. High Throughput. Screen., vol. 7, no. 3, pp. 223-229). High sensitivity is achieved by a dramatic reduction of background yielding a high signal-to noise ratio. In addition, only minimal amounts of labeled detection antibodies are applied in contrast to the direct labeling approach were a huge amount of labeled proteins are present in a sample. The sandwich immunoassay format can also be easily ameanable to the field of microarray technology, and such immunoassays can be applied to the protein microarray format to quantify proteins in conditioned media and/or patient sera (Huang et al. 2001; Clin. Chem. Lab Med., vol. 39, no. 3, pp. 209-214; Schweitzer et al. 2002; Nat. Biotechnol., vol. 20, no. 4, pp. 359-365).

In the direct labeling approach, all proteins in a sample are labeled with a fluorophore. Labeled proteins that bind to the protein microarray such as to an antibody microarray are then directly detected by fluorescence. An adaptation of the direct labeling approach is described by Haab and co-workers (Haab, Dunham, & Brown 2001; Genome Biol., vol. 2, no. 2, p). In this approach, proteins from two different biological samples are labeled with either Cy3 or Cy5 fluorophores. These two labeled samples are then equally mixed together and applied to an antibody microarray. This approach, for example, allows comparisons to be made between diseased and healthy, or treated and untreated samples. Direct labeling has several advantages, one of which is that the direct labeling method only requires one specific antibody to perform an assay.

Miniaturized and multiplexed immunoassays may also used to screen a biological sample for the presence or absence of proteins such as antibodies (Joos et al. 2000; Electrophoresis, vol. 21, no. 13, pp. 2641-2650; Robinson et al. 2002; Nat. Med., vol. 8, no. 3, pp. 295-301).

Accordingly, the invention provides for a composition such as for example a protein microarray comprising at least two detection or capture agents for determining the amounts of at least two biomarkers selected from the group consisting of TNF-alpha, MCP-1, IL-1ra, IL-8, IL-6, IL-1beta, and RF. Most preferably, said protein microarray comprises at least one antibody which specifically binds to IL-1ra and at least one further antibody specifically binding to IL-6; and/or at least one antibody which specifically binds to IL-1ra and at least one further antibody specifically binding to MCP-1, and/or at least one antibody which specifically binds to IL-1ra and at least one further antibody specifically binding to TNF-alpha, and/or at least one antibody which specifically binds to IL-6 and at least one further antibody specifically binding to MCP-1, and/or at least one antibody which specifically binds to IL-6 and at least one further antibody specifically binding to TNF-alpha, and/or at least one antibody which specifically binds to MCP-1 and at least one further antibody specifically binding to TNF-alpha. Even more preferably, the protein microarray according to the present invention comprises at least one antibody which specifically binds to IL-1ra, at least one further antibody specifically binding to IL-6, and at least one further antibody specifically binding to MCP-1; and/or at least one antibody which specifically binds to IL-6, at least one further antibody specifically binding to MCP-1, and at least one further antibody specifically binding to TNF-alpha; and/or at least one antibody which specifically binds to IL-1ra, at least one further antibody specifically binding to MCP-1, and at least one further antibody specifically binding to TNF-alpha. Most preferably, the protein microarray comprises at least one antibody which specifically binds to IL-1ra, at least one further antibody specifically binding to IL-6, and at least one further antibody specifically binding to TNF-alpha. In other preferred embodiment, such protein microarrays further comprise means such as for example antibodies to also determine the amounts of RF.

In a preferred embodiment of the invention, the detection or capture agents such as the antibodies are immobilized on a solid support, such as for example on a polystyrene surface. In another most preferred embodiment, the detection or capture agents are spotted or immobilized in duplicate, triplicate or quadruplicate onto the bottom of one well of a 96 well plate.

Another aspect the invention provides a method for determining whether a subject has or is likely to develop rheumatoid arthritis (RA), said method comprising determining the amounts of at least two biomarkers selected from the TNF-alpha, MCP-1, IL-1ra, IL-8, IL-6 and IL-1beta in a biological sample obtained from said subject. Another embodiment of the method of the invention further comprises the determination of the presence of erosions and/or the amount of RF in a biological sample. In a preferred embodiment increased amounts of the at least two biomarkers and optionally of RF in the sample from the subject relative to the amounts of said at least two biomarkers and optionally of RF in a control sample and/or the presence of bone erosions indicates that the subject has or is likely to develop RA or wherein the extent of the increase in the amounts of the at least two biomarkers and optionally of RF in the sample from the subject relative to a control sample and/or the extend of erosions correlate to the progression of RA. Preferably, the control sample is a sample obtained from a healthy individual.

Said increased amounts preferably refer to statistically significantly increased amounts, i.e. refer to levels that are significantly above the normal levels found in healthy individuals for these at least two biomarkers and/or found for RF. The term “significantly” or “statistically significant” refers to statistical significance and generally means a two standard deviation (SD) above normal, or higher, concentration of the marker. In preferred embodiments, at least about a 5 percent increase in the amounts of the at least two biomarkers and optionally in the amount of RF are an indication that the subject has or is likely to develop rheumatoid arthritis (RA). Preferably, the increase is at least about 6, 8, 10, 15, 20, 25, 30, 35, 40, 45 or 50 percent.

In another aspect the invention relates to a method for identifying a test substance that is likely to prevent or diminish RA in a subject, said method comprising the steps of a) determining the amounts of at least two biomarkers selected from the group consisting of TNF-alpha, MCP-1, IL-1ra, IL-8, IL-6 and IL-1beta in a biological sample obtained from said subject; b) contacting the biological sample with a test substance; and c) determining again the amounts of the at least two biomarkers selected from the group consisting of TNF-alpha, MCP-1, IL-1ra, IL-8, IL-6 and IL-1beta in the biological sample of step b); wherein an alteration in the amounts of the at least two biomarkers as measured in step b) relative to the amounts measured in step a) identifies a test substance that is likely to prevent or diminish RA. Another embodiment of the invention further comprises the determination of the presence of erosions and/or the amount of RF in a biological sample. According to a preferred embodiment of the invention, a decrease in the amounts of the at least two biomarkers—and optionally of RF—determined in step c) when compared to the amounts of the at least two biomarkers—and optionally of RF—determined in step a) indicates that the test substance is likely to prevent or diminish RA.

In another aspect the invention provides a method for following the efficiency of a treatment against RA said method comprising the steps of a) obtaining a biological sample from a subject suffering from RA before initiating said treatment, b) obtaining a biological sample after initiating said treatment from a subject suffering from RA, and c) comparing the amounts of at least two biomarkers selected from the group consisting of TNF-alpha, MCP-1, IL-1ra, IL-8, IL-6 and IL-1beta in the biological sample of steps a) and b), wherein similar amounts in the samples of steps a) and b) indicates that the treatment is not effective, and wherein an alteration in the amounts of the at least two biomarkers in the sample of step b) indicates that the treatment is effective. In another embodiment the method for following the efficiency of a treatment against RA said method comprises comparing the amounts of at least two biomarkers selected from the group consisting of TNF-alpha, MCP-1, IL-1ra, IL-8, IL-6 and IL-1beta, and RF in a biological sample obtained from a subject suffering from RA before initiating said treatment with the amounts of said at least two biomarkers in a biological sample obtained from said subject after initiating said treatment, wherein similar amounts in both biological samples indicates that the treatment is not effective, and wherein an alteration in the amounts of the at least two biomarkers in the biological sample obtained from said subject after initiating said treatment compared to the amounts determined in the sample before initiating said treatment indicates that the treatment is effective. Preferably, a decrease in the amounts of the at least two biomarkers in the sample of step b), i.e. in the sample obtained from the subject after initiating said treatment relative to the amount determined in the biological sample obtained from said subject before initiating said treatment indicates that the treatment is effective. Another embodiment of the invention further comprises the determination of the presence of erosions and/or the amount of RF in the biological sample. According to a preferred embodiment said treatment comprises administration of a disease-modifying anti-rheumatic drug (DMARD).

Preferably said decrease determined in one of the methods of the inventions is a statistically significant decrease, i.e. refers to levels that are significantly below levels found in RA patient for the at least two biomarkers and optionally for RF, and approximate to the levels found in healthy individuals. For example, an about 5 percent decrease in the amounts of the at least two biomarkers indicates that the test substance is likely to prevent or diminish RA or indicates that the treatment is effective. Preferably, the decrease is at least about 6, 8, 10, 15, 20, 25, 30, 35, 40, 45 or 50 percent.

Still a further aspect of the invention provides a method for identifying subjects having or likely to have a specific subtype of RA said method comprising the steps of a) obtaining a biological sample from a subject suffering from RA, b) comparing the amounts of at least two biomarkers selected from the group consisting of TNF-alpha, MCP-1, IL-1ra, IL-8, IL-6 and IL-1beta in the biological sample of steps a) with the amounts of a specific RA subtype, wherein similar amounts in the samples of steps a) and b) indicates that the subject belongs said specific subtype, and wherein a difference in the amounts of the at least two biomarkers of step a) and step b) indicates that the subject belongs to a different subtype of RA. Another embodiment of said method further comprises the determination of the presence of erosions and/or the amount of RF in the biological sample. Preferably said difference is a statistically significant difference, i.e. refers to levels of the at least two biomarkers, and optionally of RF and/or erosions that are significantly different from the levels of said biomarkers known for a specific subtype of RA.

Another aspect of the invention provides a method for identifying a subject as having or likely to have a rheumatic disease said method comprising determining the amounts of at least two biomarkers selected from the group consisting of TNF-alpha, MCP-1, IL-1ra, IL-8, IL-6 and IL-1beta in a biological sample obtained from said subject. A further embodiment of said method provides the additional determination of the presence of erosions and/or the amount of RF in the biological sample, in a preferred embodiment increased amounts of the at least two biomarkers, and optionally of RF in the sample from the subject relative to a control sample of a healthy individual, and/or the presence of erosions indicates that the subject has or is likely to develop said a rheumatic diseases. Said increased amounts comprise preferably statistically significant amounts. In preferred embodiments, at least about a 5 percent increase in the amounts, of the at least two biomarkers are an indication that the subject has or is likely to develop the rheumatic disease. Preferably, the increase is at least about 6, 8, 10, 15, 20, 25, 30, 35, 40, 45 or 50 percent. Rheumatic diseases may for example be polymylagia rheumatica (PMR), juvenile rheumatoid arthritis (JRA), connective tissue disease (CTD) and giant cell arteritis.

The determination of the amounts of the at least two biomarker—and optionally of the amount of RF and/or erosions—are preferably performed as described herein. For example, erosions may be visually and qualitatively assessed by e.g. inspection of Xray films or may be determined by using MRI analysis; the amounts of the at least two biomarkers or the amount of RF may be determined by measuring the amount of the mRNAs encoding said biomarkers using sequence-specific oligonucleotide. In another preferred embodiment said amounts of biomarkers and optionally of RF are determined by means of mass spectrometry or by means of a reagent which specifically binds to the at least two biomarker proteins and/or to RF, such as for example, antibodies, including monoclonal antibodies, antibody derivatives or an antibody fragments. Preferably said means used for the determination of the amounts of the at least two biomarkers are immobilized on a solid support, such as for example on a polystyrene surface. Most preferably, the antibodies specifically binding to the at least two biomarkers of the invention are those described in the Examples further below.

RF may be measured using different methods; all of them are based on detecting IgM RF in serum. The methods which may be used are for example: a) Waaler Rose: a classic semiquantitative assay using sheep erythrocytes coated with rabbit IgG. (Waaler E. On the occurrence of a factor in human serum activationg the specific agglutination of sheep blood corpuscles. ActaPathol. Microbiol. Scan, 1940; 17:172-173; Rose H M, Ragan C, Pearce E, Lipman M O. Differential agglutionation of normal and sensitized sheep erythrocytes by sera of patients with rheumatoid arthritis. Proc. Soc. Exp. Biol. Med. 1949; 68:1-11); b) Latex Fixation Assay: also a classic semiquantitative assay, using latex beads coated with human IgG (Singer J M, Plotz C M. The Latex Test, I. Application to the serologic diagnosis of rheumatoid arthritis. Am J Med 1956: 21:888) and/or c) Nephelometry: Automated detection using latex beads coated with human IgG. Measured on nephelometer “BNII” using “N Latex RF” reagents (device and assay by Dade Behring, OUUA G13 E0540 135 H; Weinblatt, M E, Schur P H. Rheumatoid factor detection by nephelometry. Arthitis and Rheumatism 1980; 23:777-779.

In a preferred embodiment the amount of TNF-alpha and the amount of at least one further biomarker selected from the group consisting of MCP-1, IL-1ra, IL-8, IL-6 and IL-1beta are determined. Most preferably, the at least one further biomarker is MCP-1, IL-1ra, IL-8, IL-6 or IL-1beta. In another preferred embodiment, additionally the amount of RF and/or the presence of erosions are determined.

In another preferred embodiment the amount of MCP-1 and the amount of at least one further biomarker selected from the group consisting of TNF-alpha, IL-1ra, IL-8, IL-6 and IL-1beta are determined. Most preferably, said at least one further biomarker is TNF-alpha, IL-1ra, IL-8, IL-6 or IL-1 beta. According to another preferred embodiment, the amounts of MCP-1 and TNF-alpha are determined, in other preferred embodiments, further the amount of RF and/or the presence of erosions are determined.

In a still further embodiment, the amount of IL-1ra and the amount of at least one further biomarker selected from the group consisting of TNF-alpha, MCP-1, IL-8, IL-6 and IL-1beta are determined, wherein most preferably, said at least one further biomarker is TNF-alpha, MCP-1, IL-8, IL-6 or IL-1beta, in a most preferred embodiment the amounts of IL-1ra and IL-6 are determined. In another most preferred embodiment of the Invention, the amounts of IL-1ra and MCP-1 or the amounts of IL-1ra and TNF-alpha are determined. In other preferred embodiments, in addition the amount of RF and/or the presence of erosions are determined.

In another preferred embodiment, the amount of IL-8 and the amount of at least one further biomarker selected from the group consisting of TNF-alpha, MCP-1, IL-1ra, IL-6 and IL-1beta are determined. Most preferably the at least one further biomarker is TNF-alpha, MCP-1, IL-1ra, IL-8 or IL-1beta.

In a still further embodiment the amount of IL-6 and the amount of at least one further biomarker selected from the group consisting of TNF-alpha, MCP-1, IL-1ra, IL-8 and IL-1beta are determined. Preferably, the at least one further biomarker is TNF-alpha, MCP-1, IL-1ra, IL-8, or IL-1beta. In a most preferred embodiment the amounts of IL-6 and MCP-1 or the amounts of IL-6 and TNF-alpha are determined. In other preferred embodiments, further the amount of RF and/or the presence of erosions are determined.

In another preferred embodiment, the amount of IL-1beta and the amount of at least one further biomarker selected from the group consisting of TNF-alpha, MCP-1, IL-1ra, IL-8 and IL-6 are determined. Preferably, the at least one further biomarker is TNF-alpha, MCP-1, IL-1ra, IL-8, or IL-6. In further preferred embodiments, the amount of RF and/or the presence of erosions are determined in addition.

According to a further still preferred embodiment, the amounts of at least 3 biomarkers selected from the group consisting of TNF-alpha, MCP-1, IL-1ra, IL-8, IL-6 and IL-1beta are determined. Most preferably the amounts of TNF-alpha, IL-1ra, and IL-6 are determined. In a most preferred embodiment the amounts of TNF-alpha, IL-1ra and MCP-1, of TNF-alpha, MCP-1 and IL-6, or the amounts of IL-1ra, IL-6 and MCP-1 are determined. A still further embodiment provides that the amounts of at least 4 biomarkers selected from the group consisting of TNF-alpha, MCP-1, IL-1ra, IL-8, IL-6 and IL-1beta are determined. In the most preferred embodiment of the invention the amounts of TNF-alpha, MCP-1, IL-1ra and IL-6 are determined. In another most preferred embodiment of the invention the amount of TNF-alpha, MCP-1, IL-1ra, IL-6 and of RF are determined, optionally together with the determination of bone erosion.

In another embodiment of the invention relates to a method in which the amounts of at least 5 biomarkers selected from the group consisting of TNF-alpha, MCP-1, IL-1ra, IL-8, IL-6 and IL-1beta is determined. Preferably, the amount of TNF-alpha, MCP-1, IL-1ra, IL-8 and IL-6 are determined. Further preferred embodiments provide that the amounts of no more than 4, 5 or 6 biomarker selected from the group consisting of TNF-alpha, MCP-1, IL-1ra, IL-8, IL-6 and IL-1 beta are determined. In a preferred embodiment, the amounts of TNF-alpha, MCP-1, IL-1ra, IL-8, IL-6 and IL-1beta are determined, According to other preferred methods of the invention, the amounts of the at least 2, 3, 4, 5 or no more than 4, 5 or 6 biomarkers as outlined above are measured together with the amount of RF in a biological sample and/or together with the determination of the presence of bone erosions

In a preferred embodiment the amounts of the at least two biomarkers selected from the group consisting of TNF-alpha, MCP-1, IL-1ra, IL-8, IL-6 and IL-1beta are separately, sequential or simultaneously determined, optionally together with the amount of RF. Most preferably, the amounts of the at least two biomarkers are simultaneously determined. Accordingly, the at least two biomarkers selected from the group consisting of TNF-alpha, MCP-1, IL-1ra, IL-8, IL-6 and IL-1beta, and optionally RF are simultaneously determined within one biological sample by contacting the sample with a mixture of means able to specifically bind said biomarkers such as antibodies. In another preferred embodiment of the invention the simultaneous determination of the amounts of the biomarkers is performed by using a composition or kit as described herein.

The invention is further based on the discovery of compositions for the prediction, diagnosis or prognosis of rheumatoid arthritis (RA) comprising at least two detection agents for determining the amounts of at least two biomarkers selected from the group consisting of TNF-alpha, MCP-1, IL-1ra, IL-8, IL-6 and IL-1beta. Another embodiment of said composition further comprises at least one detection agent for determining the amounts RF. Said compositions may also be used to diagnose or to monitor the progression or treatment of RA. The detection agents preferably comprise means specifically binding to TNF-alpha and at least one further means specifically binding to at least one further biomarker selected from the group consisting of MCP-1, IL-1ra, IL-8, IL-6 and IL-1beta. Most preferably said means specifically binds to TNF-alpha and said other means specifically binds to MCP-1, IL-1ra, IL-8, IL-6 or IL-1beta. In a further preferred embodiment of the invention, the detection agents comprise means specifically binding to MCP-1 and at least one further means specifically binding to at least one further biomarker selected from the group consisting of TNF-alpha, IL-1ra, IL-8, IL-6 and IL-1beta. Preferably, said at least one further biomarker is TNF-alpha, IL-1ra, IL-8, IL-6 or IL-1beta. In another embodiment of the invention the composition comprises at least one means specifically binding to MCP-1 and one further means specifically binding to TNF-alpha. In another embodiment said composition further comprises at least one means specifically binding to RF.

In a still further preferred embodiment the detection agents comprise means specifically binding to IL-1ra and at least one further means specifically binding to at least one further biomarker selected from the group consisting of TNF-alpha, MCP-1, IL-8, IL-6 and IL-1beta. Most preferably, the composition comprises at least one means specifically binding to IL-1ra and at least one further means which binds to IL-6, or at least one means specifically binding to IL-1ra and at least further means specifically binding to MCP-1, or at least one means specifically binding to IL-1ra and at least one further means specifically binding to TNF-alpha. In other embodiment said compositions further comprise at least one means specifically binding to RF.

In another preferred embodiment the detection agents comprise means specifically binding to IL-8 and at least one further means specifically binding to at least one further biomarker selected from the group consisting of TNF-alpha, MCP-1, IL-1ra, IL-6 and IL-1beta. Most preferably, the at least one further biomarker is TNF-alpha, MCP-1, IL-1ra, IL-6 or IL-1 beta. A still further preferred embodiment provides that the detection agents comprise means specifically binding to IL-6 and at least one further means specifically binding to at least one further biomarker selected from the group consisting of TNF-alpha, MCP-1, IL-1ra, IL-8 and IL-1beta. Preferably, said at least one further biomarker is TNF-alpha, MCP-1, IL-1ra, IL-8, or IL-1beta. Most preferably, the composition comprises at least one means specifically binding to IL-6 and at least one further means which binds to MCP-1 and optionally at least one means binding to RF; or the composition comprises at least one means specifically binding to IL-6 and at least one further means which binds to TNF-alpha and optionally at least one means binding to RF.

Another embodiment of the invention relates to a composition wherein the detection agents comprise means specifically binding to IL-1beta and at least one further means specifically binding to at least one further biomarker selected from the group consisting of TNF-alpha, MCP-1, IL-1ra, IL-8 and IL-6. Most preferably, said at least one further biomarker is TNF-alpha, MCP-1, IL-1ra, IL-8, or IL-6. And a still further embodiment of the invention relates to a composition wherein the detection agents comprise at least 3 different means specifically binding to at least 3 biomarkers selected from the group consisting of TNF-alpha, MCP-1, IL-1ra, IL-8, IL-6 and IL-1beta. Most preferably, said composition comprises at least one means specifically binding to TNF-alpha, at least one means specifically binding to IL-1ra, and at least one means specifically binding to MCP-1; or the composition comprises at least one means specifically binding to TNF-alpha, at least one means specifically binding to MCP-1, and at least one means specifically binding to IL-6; or the composition comprises at least one means specifically binding to IL-1ra, at least one means specifically binding to IL-6, and at least one means specifically binding to MCP-1; or the composition comprises at least one means specifically binding to IL-1ra, at least one means specifically binding to IL-6, and at least one means specifically binding to TNF-alpha. In other most preferred embodiments of the invention, said compositions comprising at least 3 different means specifically binding to the at least 3 biomarkers, further comprise at least one means binding to RF.

Still another embodiment of the invention provides a composition wherein the detection agents comprise at least 4 different means specifically binding to at least 4 biomarkers selected from the group consisting of TNF-alpha, MCP-1, IL-1ra, IL-8, IL-6 and IL-1beta. Most preferably, the at least 4 biomarkers comprise TNF-alpha, MCP-1, IL-1ra and IL-6. In another most preferred embodiment the composition comprising at least 4 different means specifically binding to at least 4 biomarkers further comprises at least one means binding to RF. An even more preferred embodiment of the invention provides that the detection agents comprise at least 5 different means specifically binding to at least 5 biomarkers selected from the group consisting of TNF-alpha, MCP-1, IL-1ra, IL-8, IL-6 and IL-1beta, wherein most preferably the at least 5 biomarkers comprise TNF-alpha, MCP-1, IL-1ra, IL-8 and IL-6. Another embodiment of the invention provides a composition wherein the detection agents comprise no more than 4, 5 or 6 different means specifically binding to no more than 4, 5 or 6 biomarkers selected from the group consisting of TNF-alpha, MCP-1, IL-1ra, IL-8, IL-6 and IL-1beta or no more than 6 biomarkers comprise TNF-alpha, MCP-1, IL-1ra, IL-8, IL-6 and IL-1beta. According to another embodiment of the invention the means specifically binding to a biomarker comprise at least one sequence-specific oligonucleotide which is able to bind to the mRNAs encoding said biomarker or comprise an antibody, a monoclonal antibody, an antibody derivative or an antibody fragment able to bind to the at least two biomarker/biomarker proteins selected from the group consisting of TNF-alpha, MCP-1, IL-1ra, IL-8, IL-6 and IL-1beta—as described in detail further above and also outlined in the Examples, in a particularly preferred embodiment the means specifically binding to a biomarker are immobilized on a solid support such as for example a polystyrene surface. A most preferred embodiment provides at least 2, 3, 4, or at least 5 or 6 antibodies specifically binding to TNF-alpha, MCP-1, IL-1ra, IL-8, IL-6 or IL-1beta, or RF as described in the Examples further below, wherein said antibodies being spotted or immobilized on a polystyrene surface, such within one well of a 96 well plate. Another preferred embodiment provides that at least two antibodies specifically binding to the at least two biomarkers selected from the group of TNF-alpha, MCP-1, IL-1ra, IL-8, IL-1beta and IL-6, and optionally RF are spotted or immobilized in duplicate, triplicate or quadruplicate within one well of a 96 well plate.

Other aspects of the invention also provide diagnostic or drug discovery kits comprising any one of the composition according to the invention and instructions for use. In a preferred embodiment said diagnostic or drug discovery kit further comprises a mixture of labeled antibodies such as for example biotinylated antibodies able to specifically bind to the at least two biomarkers selected from the group consisting of TNF-alpha, MCP-1, IL-1ra, IL-8, IL-6 and IL-1 beta, and to RF.

And a still further aspect of the invention relates to the use of such diagnostic or drug discovery kit in one of the methods according to the invention.

The Invention is further described in the Examples below.

### EXAMPLE 1

### Development of Protein Microarray in a Glass Chip Format

Protein microarrays continue to be developed as a useful tool for multiplexed protein analysis. Protein microarray development combines many technologies for protein deposition, assay procedures, signal detection and data analysis. Many attempts have been undertaken for protein microarray development that involved different supports, liquid handling and detection systems (Kodadek 2001; Chem. Biol., vol. 8, no. 2, pp. 105-115; Stoll, Templin, Schrenk, Traub, Vohringer, & Joos 2002; Front Biosci., vol. 7, p. c13-c32; Templin et al. 2003; Proteomics., vol. 3, no. 11, pp. 2155-2166). The development covers following steps:
comparison of the treatment of a glass chip with two surface chemistries: Poly-L-lysine versus self-assembled monolayer of octadecyl phosphoric acid ester.
comparison of contact and non contact piezoelectric arrayers for antibody deposition.
comparison of different concentration of coating antibodies.
comparison on different detection systems: CCD camera versus fluorescent scanner.
   The development of a sandwich protein microarray on a glass chip is described for example in Urbanowska et al. 2003: Cell Biology and Toxicology 19: 189-202.

### Assay Optimization

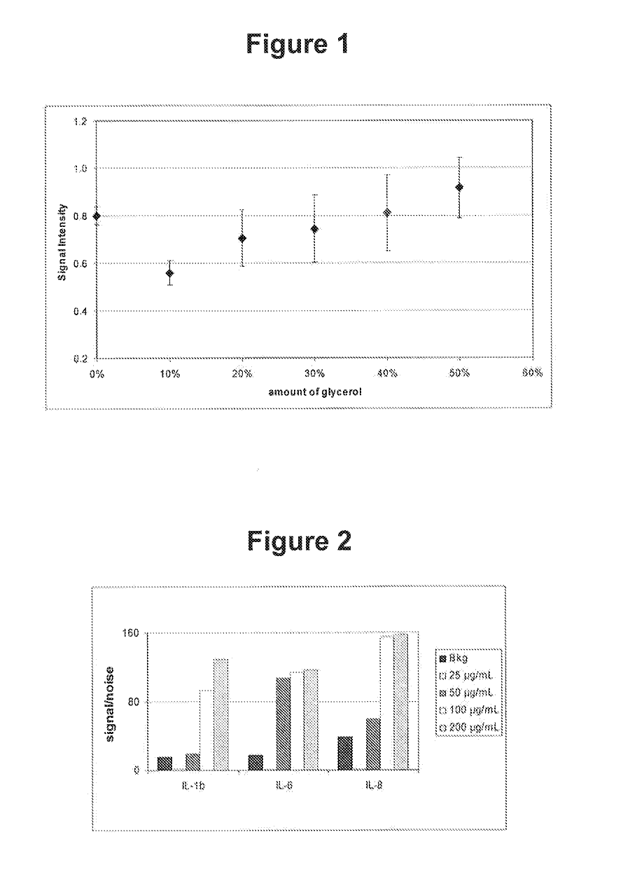

The influence of glycerol addition into a printing buffer is investigated on the MCP-1 assay. Antibodies against MCP-1 are diluted in PBS alone and in PBS with addition of 10, 20, 30, 40 and 50% of glycerol. Signal generated during sandwich assay for the concentration of 5 ng/mL of MCP-1 is plotted against different amounts of glycerol added into a coating solution (FIG. 1). The results show that the signal intensity decreased when 10% of glycerol is added into a coating solution. In case of addition of 20-50% of glycerol the generated signal is comparable however the variability between the spots (n=16) increased considerably.

### EXAMPLE 2

### Development of Protein Microarray in 96-Well Plate Format

The rather time consuming and complicated process of coating the chip (see also Example 1) with the surface chemistry, together with the lack of robotic automation for assay processing, and the high inter chip variability prompted the search for less complicated and more robust alternatives for protein microarray fabrication. Consequently an attempt was undertaken to develop a similar microarray, but instead of using glass slides, the polystyrene surface of a 96-well plate was used as the solid support.

Polystyrene plates in a 96-well format are strongly established solid supports used for immunoassays, and are widely utilized in the classical ELISA technique. The surface chemistry is standardized and optimized for antibody binding, and the assay processing step is fully automated. Therefore, the possibility to adapt this technology to the microarray format could offer many advantages over other solid supports.

The availability of long neck tips (from PerkinEimer) compatible with the Biochip Arrayer (BCA) printing device provided the option of using a 96-well plate as the solid support. Importantly, these long neck tips allow the antibodies to be correctly spotted onto the bottom of a 96-well plate (Moody, Van Arsdell, Murphy, Orencole, & Burns 2001; Biotechniques, vol. 31, no. 1, pp. 186-4). The development process involved reagent selection, printing protocol optimization, matrix investigation, assay protocol establishment, and detection system evaluation.

Based on the obtained results, the following conditions were chosen for preparation of the microarray: SAM of ODP for glass treatment; deposition of coating antibody at the concentration of 200 μg/mL using a non-contact piezoelectric printer, and signal detection by fluorescence.

As an outcome of the performed experiments 4×4 microarrays were developed in each well of a 96-well plate. Antibodies were immobilized into each well at the concentration of 200 μg/mL, 3 nL per spot using the piezoelectric device. Following a sample incubation of the mixture of antigens and biotinylated antibodies, chemiluminescent signal generated at each spot within each well was imaged using a CCD camera with non parallax tray. The image was then analyzed using ArrayVision™ software. The amount of signal generated in each spot was proportional to the amount of target protein in the original standard or sample.

### Materials and Methods

### General Reagents

PBS buffer 10× PBS buffer, Roche Diagnostic, Indianapolis, Ind., USA
Tween® 20 Fluka Chemie Sarl, Buchs, Switzerland
SuperBlock™ Pierce Biotechnology, Inc., Rockford, Ill., USA
Bovine serum albumin (fraction V) Fluka Chemie Sarl, Buchs, Switzerland
Wash buffer PBS 1× containing 0.05% Tween
Assay buffer PBS 1× containing 0.05% Tween, 3% BSA

### Software

Quantity One Version 4.2.1, Bio-Rad, Hercules, Calif., USA.
Aida Version5.0, Raytest GmbH, D 75339 Straubenhardt, Germany.
ImaGene™ Version 5.0, BioDiscovery, Inc. El Segundo, Calif., USA.
Array Vision™ Version 8.0, Imaging Research Inc., Ontario, Canada.
SOFTmax® PRO Version 3.1.1, Molecular Devices Corp., Sunnyvale, Calif., USA.

### Laboratory Equipment

Black plate: Maxisorp™ 96-well plate (Nalge Nunc International, Rochester, N.Y., USA).
Transparent plate: Maxisorp™ 96-well plate (Nalge Nunc International, Rochester, N.Y., USA).
Plate's shaker: Wesbart (IS89), Fischer scientific, Wohlen, Switzerland.
Washer: Embla, Molecular Devices, Bucher Biotec AG, Basel, Switzerland.

### Instruments

Printing instrument Biochip Arrayer (BCA) (PerkinElmer, Boston, Mass., USA).
Cameras LAS 1000—Fuji, Raytest GmbH, D 75339 Straubenhardt, Germany,
   LAS 3000—Fuji, Raytest GmbH, D 75339 Straubenhardt, Germany.

### Printing System Set Up

BCA was used for microarray fabrication. The parameters of piezoelectric systems were set to 110-200 volts per tip. The distance between the tip and bottom of the well was 0.5 mm. The antibodies were dispensed in duplicates in the 1 mm distance between the spots.

### Antibodies and Recombinant Proteins

### Antibody Reagents

| | | | Detection Ab |
|---|---|---|---|
| Analyte | Manufacturer | Capture Ab | (biotinylated) |
| IL-1β | Pierce Biotechnology, Inc., | M421BE | M420BB |
| | Rockford, IL, USA | | |
| IL-1ra | Biosource, Camarillo, CA, USA | AHC0112 | AHC0219 |
| IL-6 | Pierce Biotechnology, Inc., | M620E | M621B |
| | Rockford, IL, USA | | |
| IL-8 | Pierce Biotechnology, Inc., | M801E | M802B |
| | Rockford, IL, USA | | |
| TNFα | Pierce Biotechnology, Inc., | M303E | M302B |
| | Rockford, IL, USA | | |
| MCP-1 | R&D Systems, Minneapolis, | MAB679 | BAF279 |
| | MN, USA | | |

### Recombinant Proteins

| | | Recombinant |
|---|---|---|
| Analyte | Manufacturer | protein |
| IL-1β | Pierce Biotechnology, Inc., Rockford, IL, USA | RIL1B5 |
| IL-1ra | R&D Systems, Minneapolis, MN, USA | 250-RA |
| IL-6 | R&D Systems, Minneapolis, MN, USA | 206-IL |
| IL-8 | Pierce Biotechnology, Inc., Rockford, IL, USA | RIL810 |
| TNFα | Pierce Biotechnology, Inc., Rockford, IL, USA | RTNFA10 |
| MCP-1 | R&D Systems, Minneapolis, MN, USA | R-MCP1-20 |

### Commercial Kits

### ELISA Kits

| Analyte | Manufacturer | Ref no: |
|---|---|---|
| IL-1β | Pierce Biotechnology, Inc., Rockford, IL, USA | EH2IL1B |
| | R&D Systems, Minneapolis, MN, USA | HSLB50 |
| IL-1ra | R&D Systems, Minneapolis, MN, USA | DRA00 |
| IL-6 | Pierce Biotechnology, Inc., Rockford, IL, USA | EH2IL6 |
| | R&D Systems, Minneapolis, MN, USA | HS600B |
| IL-8 | Pierce Biotechnology, Inc., Rockford, IL, USA | EH2IL-8 |
| TNFα | Pierce Biotechnology, Inc., Rockford, IL, USA | EH3TNFA |
| | R&D Systems, Minneapolis, MN, USA | HSTA00C |
| MCP-1 | R&D Systems, Minneapolis, MN, USA | DCP00 |

### Antibody Selection

The reagents used for the antibody 96-well plate development were commercially available ELISA antibody match pairs against different epitopes, and corresponding antigens. The panel of biomarkers chosen for the microarray development was IL-1β, IL-1ra, IL-6, IL-8, MCP-1, TNFα.

In order to check the applicability of the above antibody natch pairs in the multiplex format the experiment investigating the potential cross reactivity was performed. All capture antibodies were spotted onto the bottom of each well and incubated with PBS spiked with a single recombinant cytokine (0.64 ng/mL of IL-1β, IL-6, IL-8, 0.58 ng/mL of IL-1ra, 0.8 ng/mL of MCP-1 and 0.038 ng/mL of TNFα) and a cocktail of biotinylated antibodies against all antigens. All analytes levels that were not spiked in the sample were below limit of quantification. Thus, the selected antibodies match pairs are specific in this microarray format.

The cross reactivity of a subset of antibodies against IL-1β, IL-6, IL-8 and MCP-1 was also investigated with the BIAcore® technology. The results demonstrated no cross reactivity between the antibodies match pairs selected for the microarray fabrication.

### Reagents Condition Optimization

For each step of an assay different conditions have to be examined. Various concentrations of coating and detection antibodies and different spot volume were compared. For the detection antibodies different concentration are required to generate the high signal-to-noise ratio.

### Titration of the Coating Antibody Concentration

Antibodies against IL-1β, IL-6 and IL-8 were printed in four concentrations: 25, 50, 100 and 200 μg/mL. After performing the assay, signal generated for each analyte was divided by background value. Background was quantified from the well were no analyte was added (blank). The signal to noise ratio was calculated for the concentration of 10000, 1000 and 100 pg/mL for each analyte. On the FIG. 2 the signal-to-noise ratio for the analyte concentration of 100 pg/mL is shown. For IL-1β a signal-to-noise ratio of 1.5, 19.5 92.9 and 129.2 was measured for 10, 50, 100 and 200 μg/mL of the concentration of coating antibody. In case of IL-6 for the same concentration of coating antibodies signal-to-noise ratio was 1.0, 106.9, 113.9, 116.5; while for IL-8 2.1, 59.7, 155.1, 157.8 consequently (FIG. 2). The highest signal-to-noise ratio for all analytes was obtained when the concentration of 200 μg/mL of coating antibody was used, as a result this concentration was chosen for microarray fabrication.

### Volume of Coating Solution Per Spot

Antibodies were printed into a bottom of each well. All antibodies were printed in quadruplicate per well. Each standard curve concentration was dispensed into 2 wells. Consequently 8 spots per analyte were generated. The antibody solutions were dispensed at a volume of ˜0.333 pL per drop. Four different cases of total spot volume were investigated: 20 nL (60 drops), 10 nL (30 drops), 3 nL (9 drops) and 0.333 nL (1 drop). The coefficient of variation (CV %) was calculate for each dispensed volume. When 20 nL per drop was dispensed the size of the spots was relatively big causing spots overlap each other, which made assay results inaccurate. In three other cases CV % of signal intensities generated from each spot was calculated for all standard curve concentrations: 10, 25 50 100 and 1000 pg/mL. The best sensitivity and CV % was obtained when the volume of 3 nL per spot was dispensed. The CV % range over the all standard curve concentration is shown in (Table 1). As a result the volume of 3 nL/spot was chosen for microarray fabrication.

TABLE 1
Dispensed spot volume

| | IL-1β | IL-6 | IL-8 |
|---|---|---|---|
| Spotted volume | CV % | | |
| 0.333 nL | 35.4-76.8 | 33.4-54.6 | 11.5-105.4 |
| 3 nL | 4.7-13.3 | 10.3-37.6 | 6.1-12.0 |
| 10 nL | 18.2-25.2 | 22.3-53.3 | 11.1-30.4 |

Different volumes of coating antibody solution per spot were dispensed for each analyte. CV % was calculated on signal intensities generated from each spot (n=8) using the formula: (stdev/mean*100). The CV % was the best when 3 nL/spot volume was dispensed.

### Titration of the Biotinylated Antibody Concentration

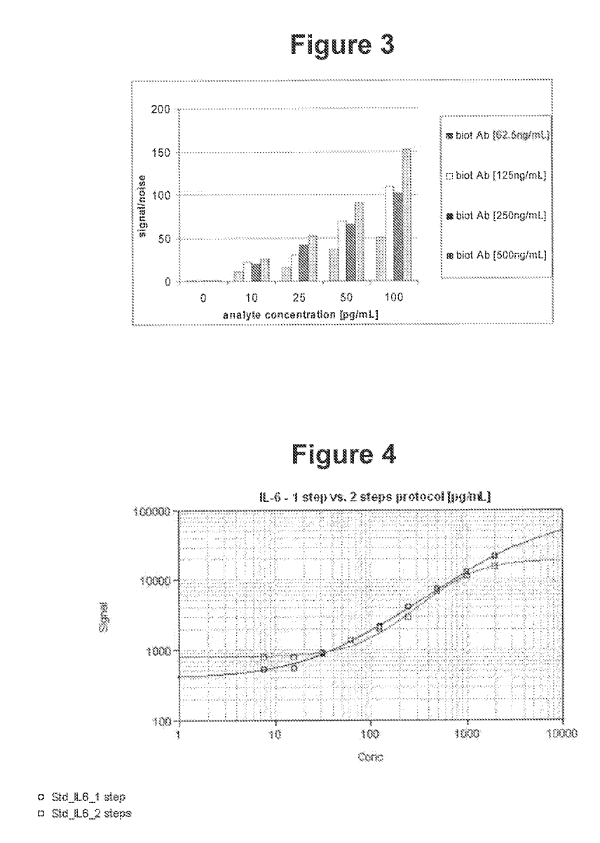

Different concentrations of biotinylated antibody were tested. Biotinylated antibody against IL-1b, IL-6 and IL-8 was diluted to 62.5, 125, 250 and 500 ng/mL. Signal generated for each dilution of biotinylated antibody was divided by corresponding background. The highest signal-to-noise ratio was obtained when the concentration of 500 ng/mL of coating antibody was used for each analyte. The example of signal-to-noise ratios generated for IL-8 assay with different concentration of biotinylated Ab is shown on FIG. 3. Consequently 500 ng/mL concentration was chosen for microarray fabrication.

### Human, Dog and Calf Serum Evaluation

Standards curves for IL-1β, IL-6 and IL-8 were prepared in random dog, human, and calf serum. Local background from each well was subtracted from the signal intensities before plotting.

### Buffer Evaluation

Standard curves for IL-1β, IL-6 and IL-8 were prepared in assay buffer used for standard generation in commercially available ELISA kits. Samples prepared from human serum pool spiked with recombinant proteins at various concentrations were calculated on the standards.

TABLE 2
Accuracies

| Spiked | Results | |
|---|---|---|
| analyte | (found analyte | |
| concentration | concentration) | Accuracy |
| ng/mL | ng/mL | % |
| 1.000 | 0.489 | 48.9 |
| 0.400 | 0.193 | 48.3 |
| 0.160 | 0.074 | 46.5 |
| 0.064 | 0.031 | 48.3 |
| 0.026 | 0.014 | 52.8 |

Accuracies [(spiked analyte concentration/found analyte concetration)*100] calculated for human serum samples spiked with IL-1β. The accuracies were calculated on IL-1β standard prepared in buffer. The obtained values gave half of the values compared to the spiked amount of IL-1β.

### Different Human Serums Screening

In order to select the matrix that contains low levels of IL-1β, IL-1ra, IL-6, IL-8, MCP-1, TNFα, antibodies against them were printed onto the bottom of each well. Different batches of human sera were applied into each well of an array. Each batch was analyzed in duplicate. Sera that gave high signal for the printed analytes were rejected and sera that generated low signal for the investigated analytes were selected as potential components of the pool. The amount of endogenous analytes in these serums was measured with the use of commercially available ELISA kits. Basing on the obtained results, serums that contained amounts of analytes corresponding to the values in normal human serum described in ELISA data sheet were selected to prepare the pool. In total 36 batches of human sera were screened from which 12 were selected and pooled together. Subsequently the amount of analytes in human serum pool was also measured with ELISA kit. The results showed that the total amount of analytes of interest In the pool also corresponds to the levels found in normal sera. The results are shown In Table 3.

TABLE 3
Analyte level in the selected human serum pool.

| | | Analyte levels range in |
|---|---|---|
| Analyte | Result [ng/mL] | normal serum [ng/mL] |
| IL-1β | blq | blq |
| IL-1ra | 0.232 | 0.106-1.552 |
| IL-6 | blq | 0-0.149 |
| IL-8 | 0.049 | 0.001-0.016 |
| TNFα | blq | blq |
| MCP-1 | 0.282 | 0.200-0.722 |

Subsequently standard curves for all analytes were produced in a human serum pool. Local background from each well was subtracted before plotting the signal and concentration. All standards showed satisfactory linearity.

Samples prepared in the same manner as during the buffer matrix evaluation were measured on the above described standards. The found concentration for all analytes corresponded to the spiked concentrations giving the assay accuracy within 70%≦ and ≦130%. The example results for IL-1β are shown in Table 4. Selected human serum pool was used for the assay validation.

TABLE 4
IL-1β assay

| | Results | |
|---|---|---|
| Spiked analyte concentration | (found analyte concentration) | Accuracy |
| ng/mL | ng/mL | % |
| 1.000 | 0.798 | 79.8 |
| 0.400 | 0.373 | 93.3 |
| 0.160 | 0.138 | 86.2 |
| 0.064 | 0.055 | 85.4 |
| 0.026 | 0.022 | 87.3 |

IL-1β human serum spike samples were measured on standard curve prepared in human serum. The concentrations were found with the satisfactory accuracy 70% ≦ and ≦130%.

### Assay Condition Optimization

Addition of Glycerol into a Coating Solution

PBS with 10, 5 and 0% addition of glycerol was used as coating solution. CV % and accuracy was calculated for all standard curve concentration points. The representative data for IL-1β is shown, in Table 5. There were no major differences in between CV % and Acc % in between the results obtained with the different addition of glycerol. Addition of glycerol however was problematic for the technical point reason. The viscous glycerol properties caused the printing tips to clog. Basing on this results PBS with no addition of glycerol was chosen as a coating buffer for microarray generation.

TABLE 5
Addition of glycerol into a coating solution.

| IL-1β | 10% glycerol | | 5% glycerol | | 0% glycerol | |
|---|---|---|---|---|---|---|
| pg/mL | CV % | Acc % | CV % | Acc % | CV % | Acc % |
| 7.8 | 7.4 | 105.1 | 13.8 | 99.7 | 11.3 | 84.0 |
| 15.6 | 17.6 | 108.4 | 19.7 | 102.8 | 26.6 | 128.9 |
| 31.2 | 15.3 | 92.4 | 12.5 | 111.5 | 24.4 | 119.8 |
| 62.5 | 16.0 | 88.4 | 37.1 | 83.5 | 27.1 | 107.3 |
| 125 | 19.7 | 99.9 | 34.0 | 94.6 | 24.0 | 92.8 |
| 250 | 16.0 | 98.9 | 28.4 | 106.0 | 16.2 | 102.2 |
| 500 | 8.7 | 108.4 | 44.8 | 108.5 | 20.3 | 99.5 |
| 1000 | 17.8 | 100.3 | 34.2 | 96.3 | 26.4 | 100.1 |

### Assay Format

Two different assay formats were compared: sandwich assay and co-incubation of the sample and biotinylated antibody. In sandwich approach assay is performed in 2 steps. Firstly printed coating antibodies are incubated with the sample, then after removing the unbound sample during the washing step the array is exposed to the cocktail of biotinylated antibodies. The 2 step incubation may be advantageous when some of assay antibodies are polyclonal.

In case of co-incubation, an assay is performed in 1 step. Printed coating antibodies are Incubated together with the mixture of sample and cocktail of biotinylated antibodies. Co-incubation approach may minimize the time difference between the sample applications. IL-6 standard curve represents the standard curves generated with the use of both assay formats (FIG. 4). When 1 step format was performed the generated standard curve was linear even in the lowest range increasing assay sensitivity.

### CCD Camera Selection and Set Up

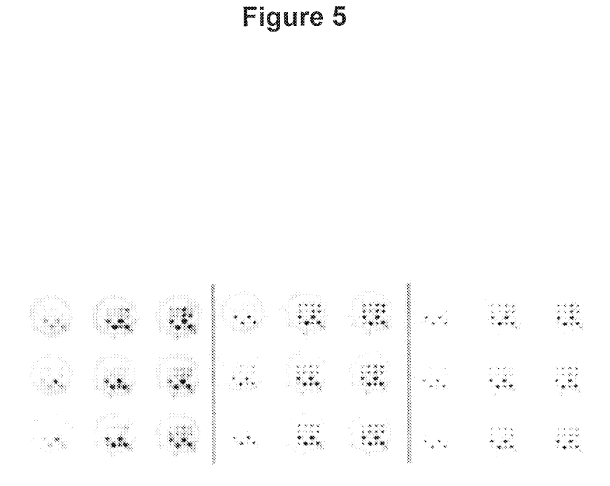

Signal visualised using HRP-conjugated streptavidin and enhanced chemiluminescence was then imaged with the use of charge-coupled device (CCD) camera. The choice of chemiluminescence and CCD combination was dictated by the 96-well format. Images generated with two different CCD cameras. The two different CCD cameras which were utilized for imaging were Fuji LAS 1000 and LAS 3000. Observed was that an image produced without non-parallax tray, created edge effect makes an image analysis imprecise. The advantage of the second camera is a non-parallax tray eliminating parallax in each of 96 well plate wells. The second camera also gave an option of high resolution binning mode which decreasing the size of the spot makes microarray analysis easier and more precise. FIG. 5 shows a picture taken with the non-parallax tray device, and with three different image modes: high (left), standard (middle) and high resolution mode (right). No more edge effect was observed. In consequence second camera was chosen for microarray fabrication. Background generated during the assay was stable over all wells of the plate. CV % between the background signal intensities from each well was 3.3%. These results indicated no need for the background correction of produced signal intensities.

### Quantification Software

Three commercially available quantification software were evaluated: Aida, Imagene and ArrayVision. The major difference between the three tools is standard curve generation feature included in ArrayVision™ system. Other programs require additional tools for standard curve generation. For instance with two other software digitized signal values had to be exported Into an Excel program to perform the variability calculations. Afterwards the data was again exported to SoftmaxPro® software for standard curve generation. This was the main factor that caused the ArrayVision™ to be chosen as the software for antibody microarray fabrication. The comparison of the three software is shown in Table 6.

TABLE 6
Quantification software comparison.

| | Aida | ImaGene | Array vision ™ |
|---|---|---|---|
| Speed | + | +++ | +++ |
| Auto spot adjustment | − | + | + |
| Easy to use | + | +++ | +++ |
| Standard curve | − | − | + |
| generation | | | |

### Developed Protocol

General reagents:

| | |
|---|---|
| Plate: | black plate |
| Coating buffer: | PBS1X |
| CCD camera: | LAS-3000. High resolution binning mode. |
| Software: | ArrayVision ® |

Assay Protocol:

| | |
|---|---|
| Coating | 200 μg/mL of monoclonal Ab |
| | 3nL/spot |
| | 2 hours drying in the Biochip arrayer enclosure |
| | over night incubation at 4° C. |
| | drying 30 minutes at RT |
| | drying 10 minutes at 37° C. |
| Blocking | −3 × 200 μL of SuperBlock/well |
| | 100 μL of SuperBlock/well - 30 minutes incubation RT |
| Samples incubation | 50 μL/well of sample |
| | 50 μL/well of cocktail of biotinylated antibodies at 500 ng/mL |
| | 4 hours incubation at room temperature on shaking platform |
| | wash 3 times with wash buffer 300 μL/well |
| Streptavidin-HRP | 100 μL/well at concentration 0.2 μg/mL |
| | 30 minutes incubation at room temperature on shaking platform |
| | wash 3 times with wash buffer 300 μL/well |
| Chemiluminescent substrate | 100 μL/well - immediate readout |
| Imaging | CCD camera exposure for 20 second |
| | images digitized and saved as 16-bit tiff files |
| Signal quantification | Arrayvision ™ |

In summary, the main components, steps and conditions in the development of an efficient method to measure simultaneously at least 2, 3, 4, 5 or 6 biomarkers associated with Rheumatoid arthritis such as TNF-alpha, MCP-1, IL-1ra, IL-8, IL-6 and/or IL-1beta in the 96-well format were evaluated. The assessment covered reagents titration, amounts of coating antibody deposited per spot, matrix selection, detection and analysis, and software evaluation.

Discussing high compared to ELISA antibodies concentrations needed to produce optimal signal-to-noise ratio the possibility of antibodies denaturation during the printing process should be considered. Another possibility is physical adsorption by which antibodies are immobilized on the polystyrene 96-well plate. Physical adsorption can lead to the protein desorption during the assay, which can lead to signal loss. When the concentration of coating solution was higher the probability of denaturation or desorption of antibodies might be lower.

Existing guidance for pharmacokinetic immunoassays recommend to prepare standard curve for analyte measurement in the matrix of the same origin as analyzed samples, alternatively in the heterologus animal matrix or in protein free buffer.

The light produced in chemiluminescence reaction is emitted equally in all directions. That is why it is recommended to use black instead of clear plates for the chemiluminescence measurements in order to avoid so called light piping phenomenon. Light piping is simply the Interference of the signal generated from each well. Black plates however require the image to be taken from above the plate which creates the danger of introducing the parallax error. Therefore two kinds of CCD camera as a imaging tool for the antibody array were compared. The non parallax fray option provided by LAS 3000 camera allowed for imaging the whole plate with no parallax effect.

### EXAMPLE 3

### Validation of Antibody Microarray in 96-Well Plate

The developed antibody microarray in 96-well plate is based on a sandwich ELISA procedure utilized in classical immunoassays (Wild 2001; The Imunoassay Handbook). Immunoassays are frequently applied for biomarkers quantification that indicates the course of the disease and antibodies elicited in response to treatment (Findlay, Smith, Lee, Nordblom, Das, DeSilva, Khan, & Bowsher 2000; J. Pharm. Biomed. Anal., vol. 21, no. 6, pp. 1249-1273). Thus, it is very important for the immunoassay to be accurate and precise. Accuracy is defined as closeness of the concentration value obtained by the method to the known true concentration of value of the analyte, while precision is a closeness of individual measures of an analyte when the method is applied repeatedly to multiple aliquots of the same biological sample.

Accuracy=(calculated concentration/nominal concentration)*100.

Precision=(standard deviation/calculated mean concentration)*100

These key characteristics of any bioanalytical method are investigated during the process of validation. Validation of an analytical method identifies the sources and quantifies the potential errors in the method (Findlay, Smith, Lee, Nordblom, Das, DeSilva, Khan, & Bowsher 2000; J. Pharm. Biomed. Anal., vol. 21, no. 6, pp. 1249-1273; Shah, Midha, Findlay, Hill, Hulse, McGilveray, McKay, Miller, Patnaik, Powell, Tonelli, Viswanathan, & Yacobi 2000; Pharm. Res., vol. 17, no. 12, pp. 1551-1557). An assay validation describes in mathematical and quantifiable terms the performance characteristics of an assay. Classical immunoassays have to be validated in line with the FDA directives (2004).

### Validation Protocol

The protocol adopted for validation of the Ab microarray was based on acceptance criteria document using the key elements of the FDA directives for pharmacokinetic immunoassays (2004). A modified protocol adopted for Ab microarray in a 96-well format was implemented for the validation process.

### Validation Protocol of Antibody Microarray in 96-Well Plate

### Matrix

Matrix is used to describe a biological medium like plasma, serum, whole blood, urine, faeces, tissue, from which the analyte(s) is quantified. All validation experiments were performed on a selected pool of human serum containing low levels of the measured analytes.

### Standard Curve

The standard curve is a relationship between instrument response and known concentration of analyte.
The linear part of standard curve should lay within the analyte concentration levels in the disease state. The standard curve was constructed using 8 different concentration levels. The samples were blanked with samples to which neither analyte(s) nor internal standard had been added.

TABLE 7
Concentrations of standard curve points.

| | | | | | | | | |
|---|---|---|---|---|---|---|---|---|
| Std 1 | Std 2 | Std 3 | Std 4 | Std 5 | Std 6 | Std 7 | Std 8 | Std9 |

ng/mL

| | | | | | | | | | |
|---|---|---|---|---|---|---|---|---|---|
| IL-1β | blank | 0.023 | 0.047 | 0.094 | 0.188 | 0.375 | 0.750 | 1.500 | 3.000 |
| IL-1ra | blank | 0.090 | 0.180 | 0.359 | 0.719 | 1.438 | 2.875 | 5.750 | 11.500 |
| IL-6 | blank | 0.023 | 0.047 | 0.094 | 0.188 | 0.375 | 0.750 | 1.500 | 3.000 |
| IL-8 | blank | 0.023 | 0.047 | 0.094 | 0.188 | 0.375 | 0.750 | 1.500 | 3.000 |
| MCP-1 | blank | 0.117 | 0.234 | 0.469 | 0.938 | 1.875 | 3.750 | 7.500 | 15.000 |
| TNF-α | blank | 0.014 | 0.027 | 0.055 | 0.109 | 0.219 | 0.438 | 0.875 | 1.750 |

Standards to in matrix freshly prepared and run on each validation day.
Standards were prepared singly and analyzed in triplicates.
Back-calculated values for the standards within the working range should give ≦30% deviation from the expected concentration (70% ≦ Accuracy ≦130%).
Maximally ¼ of the individual standards can be excluded.
At least 50% of the values (spots) at each standard concentration must be within accuracy and precision range.

### Quality Control Samples

Quality control sample (QC) is a spiked sample used to monitor the performance of a bioanalytical method and to assess the integrity and validity of the results of the unknown samples analyzed in an individual batch. QC Is prepared in the same matrix as standard the curve.
QC samples were prepared in six concentration levels for each analyte.

TABLE 8
Concentrations of quality control samples.

| | | | | | |
|---|---|---|---|---|---|
| QC1 | QC2 | QC3 | QC4 | QC5 | QC6 |

ng/mL

| | | | | | | |
|---|---|---|---|---|---|---|
| IL-1β | 0.026 | 0.064 | 0.160 | 0.400 | 1.00 | 2.500 |
| IL-1ra | 0.092 | 0.230 | 0.576 | 1.440 | 3.600 | 9.000 |
| IL-6 | 0.026 | 0.064 | 0.160 | 0.400 | 1.00 | 2.500 |
| IL-8 | 0.026 | 0.064 | 0.160 | 0.400 | 1.00 | 2.500 |
| MCP-1 | 0.128 | 0.320 | 0.800 | 2.000 | 5.000 | 12.500 |
| TNF-α | 0.015 | 0.038 | 0.096 | 0.240 | 0.600 | 1.500 |

Two QC sets were prepared from the same matrix independently for each QC concentration and analyzed in triplicates.
The QC samples should cover the anticipated dynamic concentration range, with one QC at the anticipated lower limit of quantification (LLOQ); one within 3 times the LLOQ, one approximately between the high and low QC concentrations and one close to the anticipated upper limit of quantification (ULOQ).
   LLOQ is the lowest concentration of analyte In a sample that can be quantitatively determined with suitable precision and accuracy.
   ULOQ is the highest amount of analyte in a sample that can be quantitatively determined with suitable precision and accuracy.
70% ≦ Accuracy ≦130% based on the mean value at each concentration level.
Precision ≦30% at each concentration level.
At least ⅔ of the individual QC concentration values must be within accuracy and precision range.
At least 50% of the values (spots) at each QC concentrations must be within accuracy and precision range.
Standards and QC prepared from the same stock solution.
Inter-day variability: 3 validation runs performed on different days. A validation run should be rejected only if there is an analytical problem (e.g. an error in reagents preparation, instrument failure, pipetting error or if the data from a run are so erratic that the values could only have resulted from an unexplained analytical errors).

### Stability

Stability is a physico-chemical stability of an analyte in a given solution or matrix under specific storage conditions for given time intervals.
Short-term stability of spiked samples. Stability of the stock spiking human serum pool of analyte to be evaluated at RT for at least 6 hours. After completion of the desired storage time, the stability should be tested by measuring stored vs. freshly prepared samples. The stability will be tested on QC3 and QC4.
Long-term stability: The QC3 and QC4 prepared for inter-day variability validation will be analysed on first validation run, in 1 week interval and 1 month interval.

### Standard Curve and QC Samples Preparation

The same stock solution was used for the preparation of standards and QC samples for each analyte. Standards and QC samples were freshly prepared on each analysis day from the same pool of human serum. The concentrations ranges covered for standards and QC were as indicated in validation protocol.

### Standard Curve Preparation

The pre sample solution was prepared in human serum pool using an analyte stock solution of 20 μg/mL. The stock solution was diluted two times (1:10) in human serum pool to the concentration 10 ng/mL. To prepare the target concentration of IL-1β, IL-1ra, IL-6, IL-8 and MCP-1 the 100 ng/mL concentration was used. For TNFα a concentration of 10 ng/mL was used. The cocktail of recombinant proteins in human serum was diluted 1:2 in human serum in serial dilutions to the target concentrations defined in the validation protocol. The standard curve was obtained by plotting the signal versus concentration, using logistic (ELISA) settings in ArrayVision™ for each analyte. The quality of the individual calibration lines was assessed from the accuracies and precision of the back calculated concentrations of the calibration standards. These accuracies were calculated with use of SOFTmax® PRO software.

### Calculations

Accuracy % and Precision %:

| | |
|---|---|
| Mean | of at least 50% of all spots (n = 6) for each standard |
| | concentration. |
| CV % | (SD of at least 50% of all spots for each standard concentration/ |
| | Mean) * 100%. |
| Acc % | (observed concentration/expected concentration) * 100% - |
| | based on the mean value at each concentration level. |

### QC Sample Preparation

The pre sample solution was prepared in human serum pool from the stock solution 20 μg/mL for each analyte. The stock solution was diluted two times (1:10) in human serum pool to the concentration 10 ng/mL. To prepare the target concentration of IL-1β, IL-1ra, IL-6, IL-8 and MCP-1 the 100 ng/mL concentration was used. For TNFα a concentration of 10 ng/mL was used. The cocktail of recombinant proteins in human serum was diluted 1:2.5 in serial dilutions to the target concentrations defined in the validation protocol. Calculating the inter-day accuracy and precision and the intra-day accuracy and precision of the QC samples analyzed together with calibration samples on each day (n=3) assessed the accuracy and precision of the method. QC were prepared in triplicates, 2 spots per well (n=12) for two independent QC sets. The accuracy and precision for QC samples were calculated with ArrayVision™ software using following formulas.

### Calculations

Intra-day accuracy % and precision %:

| | |
|---|---|
| Mean | of at least 50% of all spots (n = 12) for each QC concentration. |
| CV % | (SD of at least 50% of all spots for each QC |
| | concentration/Mean) * 100%. |
| Acc % | (observed concentration/expected concentration) * 100% - |
| | based on the mean value at each concentration level. |

Inter-day accuracy % and precision %:

| | |
|---|---|
| Mean | of at least 50% of all spots (n = 36) from 3 validation |
| | days for each QC concentration. |
| CV % | (SD of at least 50% of all spots from 3 validation days for each |
| | QC concentration/Mean) * 100%. |
| Acc % | (observed concentration/expected concentration) * 100% - |
| | based on the mean value at each concentration level. |

### Stability Sample Preparation

Human serum pool was spiked with the recombinant proteins at the concentrations corresponding to QC3 and QC4 for each analyte. The samples were analyzed in triplicates for two sets prepared independently. Stability of spiked human serum was measured after storage for 8 h at RT on each validation day. Fresh samples were prepared every day and stored for 8 h at RT. In case of 1 week and 1 month storage at −80° C., samples were prepared on the first validation day. The stability was investigated in single experiments after particular storage time calculating the data of stored samples on the freshly prepared standard curve.

### Assay Procedure

Experiments on each validation day were performed according to the protocol described further above.

### Calibration Curve

Standard/calibration curves were prepared in human serum pool on each validation day (total of 3 days) using the concentration of calibrants as described in the validation protocol. The signal density is defined as the integrated data value (IDV). The signal was plotted against the analyte concentration using a logistic ELISA fit. For IL-1β, IL-6, IL-8 and TNFα precision and accuracy for all calibrants were within the acceptance criteria. For the IL-1ra and MCP-1 standards, accuracy and precision for all samples except for the lowest calibration samples fulfilled the acceptance criteria. The IL-1ra lowest calibration sample, 0.090 ng/mL, gave a precision of 40.2% the accuracy met the acceptance criteria though. The MCP-1 lowest calibration sample, 0.117 ng/mL, gave an accuracy and precision of 198.3% and 38.5% respectively (Table 9 and 10).

TABLE 9
Mean accuracy of back-calculated concentrations of calibration samples.

| IL-1β | Nominal IL-1beta concentration (ng/mL) | | | | | | | |
|---|---|---|---|---|---|---|---|---|
| Day of analysis | 3.000 | 1.500 | 0.750 | 0.375 | 0.188 | 0.094 | 0.047 | 0.023 |
| Day 1 | 100.7 | 103.5 | 96.7 | 98.3 | 105.5 | 109.2 | 98.5 | 94.9 |
| Day 2 | 101 | 99.8 | 98.4 | 106.4 | 104.8 | 98.8 | 94.3 | 80.4 |
| Day 3 | 102.3 | 93.2 | 110.6 | 96.4 | 100.3 | 91.1 | 90.8 | 87.1 |
| Mean accuracy | 101.3 | 98.8 | 101.9 | 100.4 | 103.5 | 99.7 | 94.5 | 87.5 |
| IL-1ra | Nominal IL-1ra concentration (ng/mL) | | | | | | | |
| Day of analysis | 11.500 | 5.750 | 2.875 | 1.438 | 0.719 | 0.359 | 0.180 | 0.090 |
| Day 1 | 96.2 | 107.7 | 97.0 | 99.0 | 102.6 | 108.6 | 121.9 | 106.9 |
| Day 2 | 97.9 | 105.3 | 94.3 | 96.9 | 114.2 | 107.7 | 127.6 | 103.9 |
| Day 3 | 97.8 | 107.6 | 92.7 | 98.9 | 108.2 | 113.1 | 113.1 | 93.7 |
| Mean accuracy | 97.3 | 106.9 | 94.7 | 98.3 | 108.3 | 109.8 | 120.9 | 101.5 |
| IL-6 | Nominal IL-6 concentration (ng/mL) | | | | | | | |
| Day of analysis | 3.000 | 1.500 | 0.750 | 0.375 | 0.188 | 0.094 | 0.047 | 0.023 |
| Day 1 | 100.1 | 100.5 | 99.0 | 101.5 | 97.8 | 103.3 | 107.8 | 108.3 |
| Day 2 | 100.8 | 100.4 | 99.7 | 98.7 | 104.7 | 98.7 | 96.6 | 114.9 |
| Day 3 | 100.4 | 98.6 | 103.4 | 98.3 | 103.6 | 96.3 | 97.8 | 104.6 |
| Mean accuracy | 100.4 | 99.8 | 100.7 | 99.5 | 102.0 | 99.4 | 100.7 | 109.3 |
| IL-8 | Nominal IL-8 concentration (ng/mL) | | | | | | | |
| Day of analysis | 3.000 | 1.500 | 0.750 | 0.375 | 0.188 | 0.094 | 0.047 | 0.023 |
| Day 1 | 100.2 | 101.7 | 97.6 | 97.9 | 103.9 | 112.4 | 109.4 | 76.3 |
| Day 2 | 100.0 | 100.7 | 98.6 | 99.8 | 103.2 | 100.4 | 101.6 | 100.2 |
| Day 3 | 100.0 | 100.0 | 100.1 | 101.2 | 99.2 | 96.0 | 100.9 | 110.0 |
| Mean accuracy | 100.1 | 100.8 | 98.8 | 99.6 | 102.1 | 102.9 | 104.0 | 95.5 |
| MCP-1 | Nominal MCP-1 concentration (ng/mL) | | | | | | | |
| Day of analysis | 15.000 | 7.500 | 3.750 | 1.875 | 0.938 | 0.469 | 0.234 | 0.117 |
| Day 1 | 102.9 | 94.4 | 107.6 | 91.2 | 89.1 | 105.7 | 128.0 | 201.0 |
| Day 2 | 89.9 | 105.0 | 96.6 | 96.1 | 114.2 | 123.1 | 121.4 | 247.5 |
| Day 3 | 101.7 | 99.8 | 101.9 | 97.3 | 90.2 | 110.9 | 86.4 | 146.4 |
| Mean accuracy | 98.2 | 99.7 | 102.0 | 94.9 | 97.8 | 113.2 | 111.9 | 198.3 |
| TNFα | Nominal TNFa concentration (ng/mL) | | | | | | | |
| Day of analysis | 1.750 | 0.875 | 0.438 | 0.219 | 0.109 | 0.055 | 0.027 | 0.014 |
| Day 1 | 100.1 | 100.3 | 99.1 | 99.2 | 102.4 | 115.2 | 106.9 | 93.1 |
| Day 2 | 100.2 | 100.2 | 100.8 | 94.1 | 110.8 | 107.1 | 101.1 | 126.5 |
| Day 3 | 100.3 | 98.9 | 101.2 | 101.3 | 104.5 | 103.9 | 99.9 | 85.8 |
| Mean accuracy | 100.2 | 99.8 | 100.4 | 98.2 | 105.9 | 108.7 | 102.6 | 101.8 |

The back-calculated values for each standard curve concentration were accessed with SOFTmax® PRO software. Mean accuracy was calculated on at least 50% of all spots (n=6) for each standard concentration.

TABLE 10
Mean accuracy of back-calculated concentrations of calibration samples.

| IL-1β | Nominal IL-1beta concentration (ng/mL) | | | | | | | |
|---|---|---|---|---|---|---|---|---|
| Day of analysis | 3.000 | 1.500 | 0.750 | 0.375 | 0.188 | 0.094 | 0.047 | 0.023 |
| Day 1 | 18.2 | 14.1 | 8.9 | 12.0 | 13.8 | 15.4 | 11.7 | 18.3 |
| Day 2 | 25.7 | 21.5 | 24.9 | 23.2 | 14.9 | 11.3 | 15.8 | 18.3 |
| Day 3 | 24.3 | 8.6 | 21.4 | 16.9 | 12.5 | 12.8 | 14.5 | 18.1 |
| Mean precision | 22.7 | 14.7 | 18.4 | 17.4 | 13.7 | 13.2 | 14.0 | 18.2 |
| IL-1ra | Nominal IL-1ra concentration (ng/mL) | | | | | | | |
| Day of analysis | 11.500 | 5.750 | 2.875 | 1.438 | 0.719 | 0.359 | 0.180 | 0.090 |
| Day 1 | 6.5 | 21.1 | 19.7 | 17.3 | 18.7 | 17.6 | 27.6 | 31.3 |
| Day 2 | 9.7 | 7.3 | 5.5 | 11.0 | 8.5 | 15.9 | 22.0 | 61.5 |
| Day 3 | 11.3 | 13.4 | 4.6 | 11.5 | 9.5 | 16.3 | 17.5 | 27.8 |
| Mean precision | 9.2 | 13.9 | 9.9 | 13.3 | 12.2 | 16.6 | 22.4 | 40.2 |
| IL-6 | Nominal IL-6 concentration (ng/mL) | | | | | | | |
| Day of analysis | 3.000 | 1.500 | 0.750 | 0.375 | 0.188 | 0.094 | 0.047 | 0.023 |
| Day 1 | 8.7 | 11.8 | 8.5 | 13.1 | 5.3 | 11.2 | 27.6 | 24.1 |
| Day 2 | 23.8 | 15.4 | 12.6 | 13.9 | 11.5 | 10.2 | 17.5 | 27.8 |
| Day 3 | 15.2 | 11.9 | 13.5 | 10.7 | 3.4 | 6.3 | 21.8 | 29.2 |
| Mean precision | 15.9 | 13.0 | 11.5 | 12.6 | 6.7 | 9.2 | 22.3 | 27.0 |
| IL-8 | Nominal IL-8 concentration (ng/mL) | | | | | | | |
| Day of analysis | 3.000 | 1.500 | 0.750 | 0.375 | 0.188 | 0.094 | 0.047 | 0.023 |
| Day 1 | 15.0 | 15.0 | 18.4 | 17.5 | 14.2 | 27.7 | 20.0 | 27.1 |
| Day 2 | 5.0 | 9.2 | 6.9 | 5.4 | 6.0 | 11.8 | 8.4 | 28.2 |
| Day3 | 3.4 | 9.7 | 9.5 | 9.1 | 12.3 | 9.9 | 22.9 | 25.7 |
| Mean precision | 7.8 | 11.3 | 11.6 | 10.7 | 10.8 | 16.5 | 17.1 | 27.0 |
| MCP-1 | Nominal MCP-1 concentration (ng/mL) | | | | | | | |
| Day of analysis | 15.000 | 7.500 | 3.750 | 1.875 | 0.938 | 0.469 | 0.234 | 0.117 |
| Day 1 | 12.6 | 4.5 | 26.2 | 19.5 | 15.4 | 4.9 | 7.8 | 23.7 |
| Day 2 | 22.7 | 21.7 | 17.1 | 14.5 | 18.8 | 13.9 | 10.6 | 42.7 |
| Day 3 | 21.0 | 14.7 | 17.1 | 18.2 | 23.8 | 21.4 | 28.3 | 49.2 |
| Mean precision | 18.8 | 13.6 | 20.1 | 17.4 | 19.3 | 13.4 | 15.6 | 38.5 |
| TNFα | Nominal TNFa concentration (ng/mL) | | | | | | | |
| Day of analysis | 1.750 | 0.875 | 0.438 | 0.219 | 0.109 | 0.055 | 0.027 | 0.014 |
| Day 1 | 4.4 | 6.9 | 10.7 | 6.3 | 5.3 | 9.2 | 10.5 | 28.3 |
| Day 2 | 6.6 | 8.8 | 6.3 | 10.7 | 12.7 | 11.3 | 17.7 | 9.5 |
| Day 3 | 4.7 | 2.9 | 4.6 | 7.2 | 8.0 | 10.2 | 11.6 | 27.7 |
| Mean precision | 5.2 | 6.2 | 7.2 | 8.1 | 8.7 | 10.2 | 13.3 | 21.8 |

The back-calculated values for each standard curve concentration were assessed with SOFTmax® PRO software. Mean precision was calculated on at least 50% of all spots (n=6) for each standard concentration.

### Quality Control Samples

The results for intra-day accuracy and precision, and inter-day accuracy and precision are shown in Table 11 and Table 12 respectively. All QC samples met the acceptance criteria for intra and inter-day assay accuracy and precision.

TABLE 11
Intra-day Accuracy. Individual accuracy of calculated concentrations
of each QC concentration for all analytes.

| QC level | Accuracy(%) | | |
|---|---|---|---|
| [ng/L] | Day 1 | Day 2 | Day 3 |

IL-1β

| | | | |
|---|---|---|---|
| 2.500 | 96.0 | 81.8 | 96.6 |
| 1.000 | 97.6 | 103.4 | 112.7 |
| 0.400 | 93.4 | 108.4 | 101.2 |
| 0.160 | 85.9 | 94.7 | 86.7 |
| 0.064 | 93.3 | 86.7 | 83.7 |
| 0.026 | 89.4 | 93.6 | 103.8 |

IL-1ra

| | | | |
|---|---|---|---|
| 9.000 | 116.1 | 122.3 | 126.1 |
| 3.600 | 113.8 | 125.3 | 115.4 |
| 1.440 | 110.2 | 114.7 | 112.6 |
| 0.576 | 111.0 | 100.4 | 117.0 |
| 0.230 | 111.5 | 111.7 | 125.3 |
| 0.092 | 128.1 | 121.6 | 112.6 |

IL-6

| | | | |
|---|---|---|---|
| 2.500 | 99.2 | 94.7 | 102.0 |
| 1.000 | 100.4 | 97.8 | 112.5 |
| 0.400 | 103.7 | 101.8 | 113.7 |
| 0.160 | 101.7 | 97.7 | 107.9 |
| 0.064 | 111.2 | 93.5 | 102.4 |
| 0.026 | 115.9 | 79.8 | 112.2 |

IL-8

| | | | |
|---|---|---|---|
| 2.500 | 95.1 | 98.0 | 98.6 |
| 1.000 | 106.4 | 100.4 | 107.8 |
| 0.400 | 106.2 | 101.7 | 114.3 |
| 0.160 | 94.3 | 95.6 | 105.9 |
| 0.064 | 94.2 | 93.9 | 105.5 |
| 0.026 | 89.1 | 100.1 | 98.4 |

MCP-1

| | | | |
|---|---|---|---|
| 12.500 | 105.5 | 98.3 | 104.9 |
| 5.000 | 89.1 | 81.5 | 107.5 |
| 2.000 | 75.5 | 82.3 | 92.4 |
| 0.800 | 78.8 | 85.0 | 94.8 |
| 0.320 | 85.5 | 105.7 | 106.6 |
| 0.128 | 127.4 | 127.2 | 112.3 |

TNFα

| | | | |
|---|---|---|---|
| 1.500 | 96.4 | 94.5 | 98.1 |
| 0.600 | 97.3 | 95.9 | 106.0 |
| 0.240 | 101.0 | 96.5 | 105.7 |
| 0.096 | 97.1 | 91.2 | 100.6 |
| 0.038 | 95.3 | 84.5 | 93.3 |
| 0.015 | 90.4 | 103.5 | 78.6 |

TABLE 12
Intra-day Precision. Individual precision of calculated
concentrations of each QC concentration for all analytes.

| QC level | Precision(%) | | |
|---|---|---|---|
| [ng/L] | Day 1 | Day 2 | Day 3 |

IL-1β

| | | | |
|---|---|---|---|
| 2.500 | 23.4 | 20.7 | 24.5 |
| 1.000 | 15.2 | 20.4 | 23.8 |
| 0.400 | 137 | 22.8 | 23.3 |
| 0.160 | 14.8 | 23.1 | 18.7 |
| 0.064 | 25.2 | 17.0 | 15.5 |
| 0.026 | 14.2 | 23.0 | 9.0 |

IL-1ra

| | | | |
|---|---|---|---|
| 9.000 | 23.4 | 21.2 | 12.4 |
| 3.600 | 15.2 | 13.9 | 13.7 |
| 1.440 | 13.7 | 17.8 | 15.7 |
| 0.576 | 14.8 | 25.5 | 20.8 |
| 0.230 | 25.2 | 22.0 | 28.5 |
| 0.092 | 14.2 | 23.7 | 24.8 |

IL-6

| | | | |
|---|---|---|---|
| 2.500 | 11.6 | 14.9 | 19.5 |
| 1.000 | 6.9 | 15.1 | 14.5 |
| 0.400 | 8.4 | 13.1 | 15.9 |
| 0.160 | 10.9 | 16.4 | 14.1 |
| 0.064 | 17.2 | 15.2 | 14.2 |
| 0.026 | 25.1 | 28.7 | 27.5 |

IL-8

| | | | |
|---|---|---|---|
| 2.500 | 12.2 | 10.3 | 9.5 |
| 1.000 | 12.0 | 6.8 | 9.6 |
| 0.400 | 12.5 | 10.7 | 14.0 |
| 0.160 | 17.7 | 14.8 | 17.5 |
| 0.064 | 23.6 | 17.8 | 26.8 |
| 0.026 | 28.1 | 26.3 | 21.7 |

MCP-1

| | | | |
|---|---|---|---|
| 12.500 | 24.6 | 27.2 | 17.1 |
| 5.000 | 19.2 | 15.4 | 22.0 |
| 2.000 | 18.7 | 18.9 | 21.7 |
| 0.800 | 17.3 | 24.1 | 27.6 |
| 0.320 | 24.8 | 24.8 | 28.4 |
| 0.128 | 21.1 | 3.3 | 29.8 |

TNFα

| | | | |
|---|---|---|---|
| 1.500 | 9.5 | 10.0 | 8.6 |
| 0.600 | 8.5 | 8.6 | 7.6 |
| 0.240 | 13.3 | 11.2 | 8.8 |
| 0.096 | 12.8 | 11.6 | 17.3 |
| 0.038 | 13.3 | 17.1 | 17.4 |
| 0.015 | 24.5 | 22.8 | 29.4 |

TABLE 13
Inter-day accuracy and precision. The range of accuracy and precision
over the all QC sample concentrations.

| | Accuracy % | Precision % |
|---|---|---|
| IL-1β | 87.9-104.6 | 17.5-24.3 |
| IL-1ra | 109.5-121.1 | 14.7-26.3 |
| IL-6 | 98.6-106.8 | 14.0-28.9 |
| IL-8 | 96.0-107.4 | 10.3-28.9 |
| MCP-1 | 83.4-122.1 | 21.8-28.3 |
| TNFα | 89.2-101.1 | 9.4-27.9 |

### Assay Working Range

The assay working range was determined between the LLOQ and the ULOQ. The assay working range met the disease concentrations for the measured analytes.

TABLE 14
Assay working range determined by between the LLOQ and the ULOQ.

| | |
|---|---|
| Analyte | Assay working range |

pg/mL

| | |
|---|---|
| IL-1β | 26-2500 |
| IL-1ra | 230-9000 |
| IL-6 | 26-2500 |
| IL-8 | 26-2500 |
| MCP-1 | 320-12500 |
| TNFα | 15-1500 |

### Stability

All the analytes except for MCP-1 were stable after 8 h at RT and after 1 week at −80° C. storage. After 1 month storage at −80° C., the concentration of the spiked samples were three time less when compared to the spiked nominal value. These results are shown in Table 15 for RT storage determination, and in Table 16 for one week and 1 month storage at −80° C.

TABLE 15
Short-term stability. Accuracy of calculated concentration of each
QC samples after 8 h of storage at RT. Fresh samples were prepared
on each validation day.

| | QC | Day 1 | Day 2 | Day 3 |
|---|---|---|---|---|
| Analyte | Nominal value [ng/mL] | Accuracy % | | |
| IL-1β | 0.4 | 110.2 | 88.2 | 88.0 |
| | 0.16 | 80.2 | 79.9 | 71.5 |
| IL-1ra | 1.44 | 98.7 | 102.4 | 87.6 |
| | 0.58 | 111.8 | 104.6 | 96.1 |
| IL-6 | 0.4 | 93.7 | 96.4 | 77.6 |
| | 0.16 | 95.6 | 97.6 | 84.9 |
| IL-8 | 0.4 | 112.8 | 111.9 | 88.6 |
| | 0.16 | 110.2 | 105.1 | 89.2 |
| MCP-1 | 2 | 53.5 | 70.0 | 71.0 |
| | 0.8 | 50.9 | 60.6 | 70.3 |
| TNFα | 0.24 | 106.3 | 92.0 | 80.4 |
| | 0.096 | 94.9 | 84.0 | 75.0 |

TABLE 16
Long-term stability of spiked samples. Accuracy of calculated
concentration of each QC samples after 1 week storage at −80° C.
and 1 month of storage at −80° C.

| | | After 1 week at | After 1 month at |
|---|---|---|---|
| | QC | −80° C. | −80° C. |
| Analyte | Nominal value [ng/mL] | Accuracy % | |
| IL-1β | 0.4 | 94.6 | 35.7 |
| | 0.16 | 74.1 | 24.7 |
| IL-1ra | 1.44 | 97.7 | 43.6 |
| | 0.58 | 94.6 | 35.0 |
| IL-6 | 0.4 | 101.7 | 57.3 |
| | 0.16 | 96.7 | 41.4 |
| IL-8 | 0.4 | 97.5 | 25.1 |
| | 6.16 | 87.2 | blq |
| MCP-1 | 2 | 45.9 | 27.0 |
| | 0.8 | 23.6 | 41.4 |
| TNFα | 0.24 | 89.7 | 53.7 |
| | 0.096 | 80.3 | 31.9 |

Immunoassay as a technology used for biomarker quantification needs to be validated in terms of accuracy, precision and reproducibility. The antibody microarray in 96-well format was validated based on the Food and Drug regulatory guidelines for pharmacokinetic assays. The following parameters were investigated during the validation process: calibrants accuracy and precision based on back calculated values; intra and inter day accuracy and precision based on quality control samples; stability of antigen spiked human serum samples after 8 h storage at RT as well as after 1 week and 1 month storage at −80° C.; assay WR based on LLOQ and ULOQ.

The obtained data show that accuracy and precision for all calibration samples for IL-1β, IL-6, IL-8, and TNFα analytes met the acceptance criteria. In case of MCP-1 accuracy and precision and for IL-1ra only precision for the lowest concentration of standard curve did not covered the acceptance criteria. However these samples are excluded during the assay working range determination. Overall the results are satisfactory because the assay working range covers the examined analytes levels in RA serums.

Validation results demonstrate that antibody microarray in 96-well plate is accurate, precise and reproducible within the determined assay working range.

### EXAMPLE 4

### Comparison of Antibody Microarray and ELISA Technology

ELISA is a standard method for analyzing protein levels in serum samples. As easy to perform and specific method, ELISA has been broadly used for the detection of serum cytokines in clinical and microbiological research over the last decade (Klimiuk et al. 2002; Lloyd et al. 1991; Ann. Rheum. Dis., vol. 61, no. 9, pp. 804-809; Mangge et al. 1995; Arthritis Rheum., vol. 38, no. 2, pp. 211-220). In this section protein microarray validated as described above is compared with the ELISA technology. IL-1β, IL-1ra, IL-6, IL-8, MCP-1 and TNFα levels were measured in 35 RA samples, 14 rheumatic diseases samples and in 26 control human serums using both technologies. Subsequently the results obtained with both methods were compared using linear regression analysis.

Multiplex Assay for IL-1β, IL-1ra. IL-6, IL-8, MCP-1 and TNFα

The serum concentrations of IL-1β, IL-1ra, IL-6, IL-8, MCP-1 and TNFα were measured with protein microarray validated as described above.

### ELISA Assay for IL-1β, IL-1ra, IL-6, IL-8, MCP-1 and TNFα

Sandwich ELISA was prepared with the same reagents that were used for microarray fabrication and for antibodies matched pairs and recombinant proteins respectively. Serum pool was used to prepared standard curves and QC samples with the concentration points the same as used to produce antibody microarray (Table 7 and Table 8). ELISAs were performed according to the manufacturer's instructions.

### Spiked Sample Preparation

Human serum pool was spiked with recombinant Il-1β, IL-6, IL-8 and TNFα and the concentration of 3, 1.500, 0.750, 0.375, 0.188, 0.094, 0.047, 0.023, 0.012 and 0.006 ng/mL. The concentration were chosen in the manner to cover the standard curve range of microarray and ELISA used for cytokine level determination. The samples were frozen at −80° C.

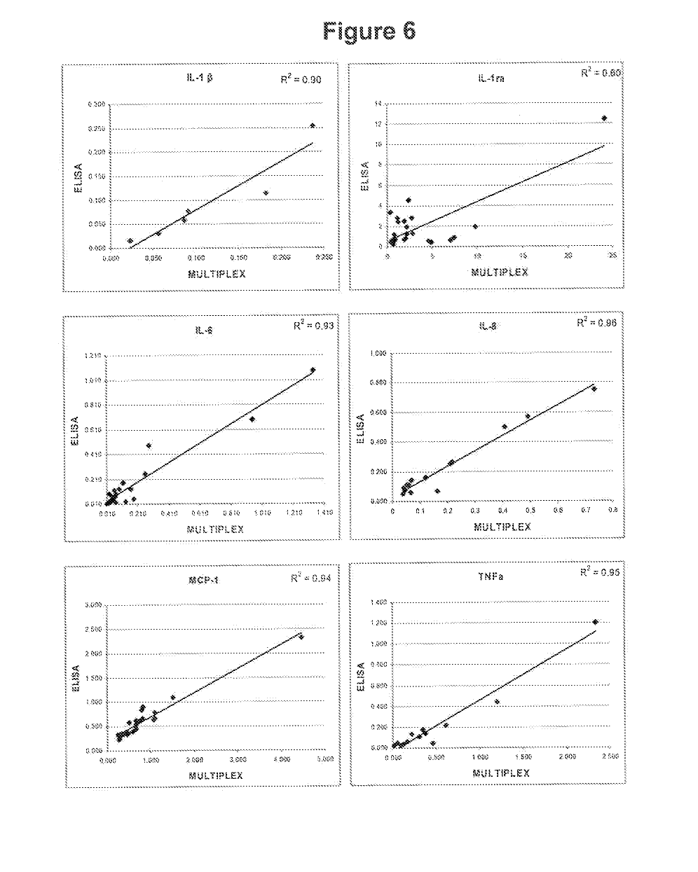

Multiplex assay performance was compared with sandwich ELISA assay. 52 rheumatic patients serum samples and 26 random human serums were assayed with both multiplex assay and sandwich ELISA. The results from two analysed groups were pooled together to make a group of 78 sera, in case of IL-1β assay the number of detectable values by both technologies were too small to perform regression analysis. In order to perform correlation analysis human serum pool was spiked with 10 different concentrations of all analytes of interests. The levels of IL-1β, IL-6, IL-8 and TNFα were also measured in the spiked samples. The results from spiked samples were pooled with the results from the 78 sera analyzed previously. Analytes concentration for 78 for all analytes and 10 spiked samples for IL-1β, IL-6, IL-8 and TNFα were subjected to linear regression analysis. The results are shown in FIG. 6. The correlation coefficients (R²) for IL-1β, IL-1ra, IL-6, IL-8, MCP-1, and TNFα were 0.90, 0.60, 0.93, 0.96, 0.94 and 0.95 respectively.

The multiplex assay for IL-1β, IL-6, IL-8, MCP-1 and TNFα showed very good correlation with individual ELISAs for the same analytes. The correlation coefficients were 0.90, 0.93, 0.96, 0.94 and 0.95 respectively. For IL-1ra assay the correlation with ELISA was lower (0.60). This could be explained by the problems often met with sandwich immunoassay and possibly magnified by assay multiplexing. For instance rheumatoid factors or naturally occurring human heterophilic antibodies often associated with autoimmune or inflammatory diseases, which can recognize animal and human immunoglobulin. Consequently false-positive or -negative results are generated (Hennlg, Rink, Fagin, Jabs, & Kirchner 2000; J. Immunol. Methods, vol. 235, no. 1-2, pp. 71-80). Another reason could be the presence of soluble receptor IL-1 Type I (Arend 2002; Cytokine Growth Factor Rev., vol. 13, no. 4-5, pp. 323-340). Maybe the binding affinity of IL-1ra to its soluble receptor is biased by multiplex format. That can also cause false high or low cytokine level different for multiplex and single ELISA.

Good correlation between protein microarray and ELISA shows the applicability of protein microarray to monitor several analytes per sample and its potential as high throughput screening tool.

### EXAMPLE 5

### Application of Antibody Microarray to Evaluate Informative Analytes in Rheumatic Samples

The multiplex assay was used to measure IL-1β, IL-1ra, IL-6, IL-8, MCP-1 and TNFα serum concentrations in samples from the patients diagnosed with rheumatoid arthritis (RA) and other rheumatic diseases.

### Subjects and Samples

Studied samples consisted of 35 samples from patients diagnosed with RA and 13 samples from patients diagnosed with rheumatic diseases such as polymylagia rheumatica (PMR), juvenile rheumatoid arthritis (JRA), connective tissue disease (CTD) and giant cell arteritis. Characteristics of the patient's populations are shown in Table 17.

TABLE 17
Patient's characteristics. Data presented as mean ± SD. Patients that were
previously treated with DMARDS are considered as DMARDS untreated
patients.

| | RA (n = 35) | Non RA (n = 13) |
|---|---|---|
| Women/men | 29/6 | 8/5 |
| Age (years) | 61.8 ± 15.1 | 58.6 ± 14.5 |
| Disease duration (years) | 9.7 ± 9.2 | NA |
| RF positive patients | 22/35 | 2/13 |
| DMARDS treated patients | 34/35 | 7/13 |

SD—standard deviation
RF—rheumatoid factor
UMARDS—disease modifying anti-rheumatic drugs
NA—not assessed

Information about patient's extra-articualar manifestations (bone erosions, skin changes, pleuropulmonary manifestations, heart disease, Raynaud's phenomenon, Sjörgen's syndrome, rheumatoid vasculitis, osteoporosis and major joint replacement) were also collected.

The serum concentrations of IL-1β, IL-1ra, IL-6, IL-8, MCP-1 and TNFα were measured with the protein microarray validated as described further above.

Analysis of the data was performed using one way Anova analysis (Kempthorne O 1983). The results were considered significant when the p values was less the 5% (p<0.05).

Measured serum levels of IL-1β, IL-1ra, IL-6, IL-8, MCP-1 and TNFα were analysed in two steps. Firstly the mean values for each of the six analytes were calculated for the RA and non RA samples. These data showed the difference for 5 analytes levels (IL-1ra, IL-6, IL-8, MCP-1 and TNFα) between the two groups (Table 18).

TABLE 18
Serum concentrations of IL-1β, IL-1ra, IL-6, IL-8, MCP-1 and TNFα for RA versus
non-RA patients. Data is presented as mean and log (mean) and their SD respectively.

| | RA | | | | Non RA | | | |
|---|---|---|---|---|---|---|---|---|
| | MEAN | SD | LOG | LOG | MEAN | SD | LOG | LOG |
| Analyte | [ng/ml] | [ng/ml] | (MEAN) | (SD) | [ng/ml] | [ng/ml] | (MEAN) | (SD) |
| IL-1β | 0.000 | 0.000 | −3.834 | −3.585 | 0.000 | 0.001 | −3.394 | −3.101 |
| IL-1ra | 1.074 | 2.160 | 0.031 | 0.334 | 0.140 | 0.112 | −0.853 | −0.950 |
| IL-6 | 0.090 | 0.160 | −1.045 | −0.796 | 0.004 | 0.007 | −2.447 | −2.162 |
| IL-8 | 0.070 | 0.162 | −1.157 | −0.791 | 0.029 | 0.057 | −1.539 | −1.241 |
| IV1CP-1 | 0.551 | 0.430 | −0.259 | −0.366 | 0.243 | 0.240 | −0.615 | −0.620 |
| TNFα | 0.140 | 0.312 | −0.853 | −0.505 | 0.002 | 0.002 | −2.621 | −2.653 |

SD—standard deviation

The analysis included the comparison of three patient's groups: RA versus non RA, Rheumatoid factor (RF) positive versus negative, bone erosion positive versus negative. The analysis were performed in regard to serum levels of six analytes of interest, age of the patient, disease duration and number of currently administered DMARDS. Disease duration is defined from the date of the diagnosis until the sample collection date.

The results of the comparison of RA and non RA rheumatic patients are shown in Table 19. The concentrations of IL-1ra, IL-6, MCP-1 and TNFα in serum were significantly higher in RA patients (p<0.0022, p<0.0107, p<0.0024 and p<0.0057 respectively). In addition the number of DMARDS was also significantly different between the investigated patient's populations (p<0.0080).

TABLE 19
One way Anova analysis for RA versus non RA patients.

| | Mean | | | | |
|---|---|---|---|---|---|
| | (non | SD | Mean | SD | p-value |
| Column ID | RA) | (non RA) | (RA) | (RA) | (RA) |
| age | 58.6154 | 14.4945 | 61.8286 | 15.1376 | 0.5121 |
| disease duration | 9.5000 | 12.0208 | 10.2857 | 9.5412 | 0.9112 |
| number of DMARDS | 0.6923 | 0.7511 | 1.3143 | 0.6311 | 0.0060 |
| log (IL-1beta) | −3.7043 | 0.4199 | −3.7741 | 0.2949 | 0.5207 |
| log (IL1-ra) | −0.9733 | 0.3361 | −0.3847 | 0.6171 | 0.0022 |
| log (IL-6) | −2.8904 | 0.6490 | −1.9610 | 1.1897 | 0.0107 |
| log (IL-8) | −1.7916 | 0.3407 | −1.5526 | 0.4979 | 0.1182 |
| log (MCP-1) | −0.8737 | 0.5469 | −0.3988 | 0.4164 | 0.0024 |
| log (TNFa) | −2.7765 | 0.3848 | −1.8638 | 1.1022 | 0.0057 |

The results from the comparison of RF positive versus RF negative patients are shown in Table 20, There was a significant difference between this two groups of patients for IL-1ra, IL-6 and TNFα (p<0.0210, p<0.0325, p<0.0109 respectively).

TABLE 20
One way Anova analysis for RF positive versus RF negative patients.

| | Mean | | | | |
|---|---|---|---|---|---|
| | (non | SD | Mean | SD | p-value |
| Column ID | RF) | (non RF) | (RF) | (RF) | (RF) |
| age | 57.1364 | 16.0899 | 63.7917 | 13.7208 | 0.2380 |
| disease duration | 9.4167 | 10.1843 | 10.1739 | 9.5948 | 0.6731 |
| number of DMARDS | 0.9545 | 0.6530 | 1.2500 | 0.7372 | 0.0818 |
| log (IL-1beta) | −3.7583 | 0.3472 | −3.7401 | 0.3323 | 0.8038 |
| log (IL1-ra) | −0.8047 | 0.4094 | −0.3316 | 0.6992 | 0.0210 |
| log (IL-6) | −2.6150 | 0.9245 | −1.9542 | 1.2258 | 0.0325 |
| log (IL-8) | −1.7425 | 0.3755 | −1.4801 | 0.5300 | 0.1174 |
| log (MCP-1) | −0.6588 | 0.5103 | −0.4005 | 0.4834 | 0.2099 |
| log (TNFa) | −2.5858 | 0.7778 | −1.7294 | 1.1213 | 0.0109 |

Table 21 shows the results of the comparison between patients with erosions and patients without erosion. There was a significant difference for IL-1ra and IL-6 between this two groups (p<0.0105 and p<0.0220). The differences were also observed in regards to disease duration (p<0.0002) and number of DMARDS (p<0.0058) between the analysed populations.

TABLE 21
One way Anova analysis for patients diagnosed with erosion (E) versus
non erosions patients (non E).

| | Mean | | | | |
|---|---|---|---|---|---|
| | (not | Std | Mean | SD | p-value |
| Column ID | E) | (not E) | (E) | (E) | (Erosions) |
| age | 62.0400 | 16.6795 | 59.7826 | 12.9192 | 0.6049 |
| disease duration | 3.4286 | 4.1826 | 14.3913 | 9.4568 | 0.0002 |
| number of DMARDS | 0.8800 | 0.6000 | 1.4348 | 0.7278 | 0.0058 |
| log (IL-1beta) | −3.7737 | 0.3331 | −3.7351 | 0.3329 | 0.6906 |
| log (IL1-ra) | −0.7566 | 0.4674 | −0.3132 | 0.6738 | 0.0105 |
| log (IL-6) | −2.5707 | 0.9646 | −1.8236 | 1.2127 | 0.0220 |
| log (IL-8) | −1.6861 | 0.3879 | −1.5425 | 0.5438 | 0.2946 |
| log (MCP-1) | −0.6119 | 0.5004 | −0.4356 | 0.4880 | 0.2235 |
| log (TNFa) | −2.3499 | 0.8622 | −1.8513 | 1.1709 | 0.0979 |

The results showed the significant difference in the serum concentration of IL-1ra, IL-6, MCP-1 and TNFα between RA and other arthritic patients. Number of DMARDS was also different between these two populations.

Studying RF positive and RF negative patient populations, the difference was observed in the levels of IL-1ra, IL-6 and TNFα.

Finally there was also a difference in the measured levels of IL-1ra and IL-6 in patients with and without bone erosions. In addition the disease duration and number of DMARDS parameters could also be distinguished between patients with or without erosions. Erosion appears in the late, more advanced stage of the disease and in more aggressive form of arthritis.

The analytes (IL-1ra, IL-6, MCP-1, TNFα) for which the differences were observed in the measured levels between the respective populations were identified to be associated with RA (Arend 2001; Arthritis Rheum., vol. 45, no. 1, pp. 101-106; Arend & Gabay 2004; Rheum. Dis. Clin. North Am., vol. 30, no. 1, p. 41-vi; Choy & Panayi 2001; N. Engl. J. Med., vol. 344, no. 12, pp. 907-916). Moreover, IL-1ra (Anakira) (Fleischmann et al. 2003; Arthritis Rheum., vol. 48, no. 4, pp. 927-934; Smolen & Steiner 2003; Nat. Rev. Drug Discov., vol. 2, no. 6, pp. 473-488) and TNFα inhibitors (entracept, infliximab and adalimumab) (Breedveld et al. 2004; Ann. Rheum. Dis., vol. 63, no. 2, pp. 149-155; Lipsky et al. 2000; N. Engl. J. Med., vol. 343, no. 22, pp. 1594-1602) belong to biological DMARDS therapies. In addition, preliminary studies involving an anti-IL-6 receptor antibody (MRA) suggest that it is approximately as effective as the TNFα directed approaches (Wendling, Racadot, & Wijdenes 1993; J. Rheumatol., vol. 20, no. 2, pp. 259-262). Also potential anti-MCP-1 treatment with monoclonal antibody to MCP-1 has been tested in arthritis models (rat collagen induced arthritis (CIA)) (Ogata et al. 1997; J. Pathol., vol. 182, no. 1, pp. 106-114).

### EXAMPLE 6

### Statistical Analysis

The raw data comprising the clinical data and concentrations of the biomarkers from the multiplex assay (Example 5) were further statistically analysed. Univariate statistical comparisons (chi-squared test for binary data and Student's t-test for continuous data) of the clinical data of rheumatoid versus non-rheumatoid patients resulted In following p-value: Centre (p=0.006), sex (p=0.23), RF (p=0.002), MTX pre-treatment (p=0.006), NSAID (p=0.38), Erosions (p=0.0008), Extraarticular (p=0.54), anti TNF-alpha treatment (p=0.62), DMARDS (p=0.0001), age (p=0.57), and duration since diagnosis (p=0.93).

Students t-test applied to the analyte concentrations of rheumatoid versus non-rheumatoid patients yielded following p-value: IL-1 beta (p-value=0.47); IL1-ra (p-value=0.02); IL-6 (p-value=0.007); IL-8 (p-value=0.23); MCP-1 (p-value=0.005); and TNF-alpha (p-value=0.02).

Multivariate statistical analysis was performed by Partial Least Squares—Discriminant Analysis (PLS-DA, SIMCA-P+ Vers 10.5) including cross-validation and validation of response permutation. Separate predictive models were applied to clinical data only (without centre, sex, age, and duration since diagnosis), analyte data only, and several combinations of both parameter sets. The predictive power of the different models were measured against the explained variance of input the variables (R2X) and the response variable (R2Y), and the fraction of total variation of the response that can be predicted by the model according to cross-validation (Q2). In addition, a Students t-test was applied to the predicted scores of the models. A ranking of the different models was based on Q2. The results of the analysis are presented in Table 22.

TABLE 22
Multivariate statistical analysis

| | | | | Scores t-test |
|---|---|---|---|---|
| Combination | R2X | R2Y | Q2 | p-value |
| IL1-RA, IL6, MCP-1; TNF-a; RF; | 0.508 | 0.374 | 0.336 | 0.000000022 |
| Erosions | | | | |
| RF; Erosions | 0.645 | 0.353 | 0.311 | 0.000000694 |
| IL1-RA, IL6, MCP-1; TNF-a; | 0.560 | 0.328 | 0.283 | 0.000000400 |
| Erosions | | | | |
| IL1-RA, IL6, MCP-1; TNF-a; RF | 0.571 | 0.302 | 0.267 | 0.000001172 |
| Erosions | 1.000 | 0.254 | 0.236 | 0.000019392 |
| IL1-RA, IL6, MCP-1; TNF-a | 0.660 | 0.240 | 0.195 | 0.000028797 |
| IL1-RA | 1.000 | 0.178 | 0.155 | 0.000300000 |
| TNF-a | 1.000 | 0.145 | 0.144 | 0.000300000 |
| MPC-1 | 1.000 | 0.180 | 0.141 | 0.010000000 |
| RF | 1.000 | 0.208 | 0.139 | 0.000840065 |
| IL6 | 1.000 | 0.121 | 0.093 | 0.003000000 |

R2X: proportion of variance in predictive parameters explained by the model
R2Y: proportion of variance in response variable explained by the model
Q2: predictive power of the model based on cross-validation Scores t-test: statistical comparison (Students t-test, two-sided, unequal variances) of the prediction scores of both groups

### LIST OF ABBREVIATIONS

Ab antibody
BCA biochip arrayer
HRP horse radish peroxidise
IL-1beta, IL-1β, IL-1b interleukin-1beta
IL-1Ra; IL-1ra IL-1 receptor antagonist
IL-6 interleukin-6
IL-8 interleukin-8
MCP-1 macrophage chemoattractant protein-1
ODP octadecyl phosphoric acid ester
RA Rheumatoid arthritis
RF rheumatoid factor
SAM self assembled monolayer
TNF-alpha, TNFα, TNF-α tumor necrosis factor-alpha

1. A method for determining whether a subject has or is likely to develop rheumatoid arthritis (RA), said method comprising determining the amounts of at least biomarkers consisting of TNF-alpha, MCP-1, IL-1ra, IL-8, IL-6 and IL-1beta in a biological sample obtained from said subject.

2. The method of claim 1, wherein increased amounts of the at least two biomarkers in the sample obtained from the subject relative to a control sample indicates that the subject has or is likely to develop RA.

3. A method for identifying a test substance that is likely to prevent or diminish RA in a subject, said method comprising
a) determining the amounts of at least two biomarkers consisting of TNF-alpha, MCP-1, IL-1ra, IL-8, IL-6 and IL-1beta in a biological sample obtained from said subject;
b) contacting the biological sample with a test substance;
c) determining again the amounts of the at least two biomarkers selected from the group consisting of TNF-alpha, MCP-1, IL-1ra, IL-8, IL-6 and IL-1beta and RF in the biological sample of step b; wherein an alteration in the amounts of the at least two biomarkers as measured in step b) relative to the amounts as measured in step a) identifies a test substance that is likely to prevent or diminish RA.

4. The method of claim 3, wherein a decrease in the amounts of the at least two biomarkers determined in step c) when compared to the amounts of the at least two biomarkers determined in step a) indicates that the test substance is likely to prevent or diminish RA.

5. A method for following the efficiency of a treatment against RA said method comprising comparing the amounts of at least two biomarkers selected from the group consisting of TNF-alpha, MCP-1, IL-1ra, IL-8, IL-6 and IL-1beta and RF in a biological sample obtained from a subject suffering from RA before initiating said treatment with the amounts of said at least two biomarkers in a biological sample obtained from said subject after initiating said treatment, wherein similar amounts in the biological samples indicates that the treatment is not effective, and wherein an alteration in the amounts biomarkers in the biological sample obtained from said subject after initiating said treatment indicates that the treatment is effective.

6. The method of claim 5, wherein a decrease in the amounts of the at least two biomarkers in the biological sample obtained from the subject after initiating said treatment relative to the amount determined in the biological sample obtained from said subject before initiating said treatment indicates that the treatment is effective.

7. The method of claim 1, wherein the biological sample is selected from the group consisting of a whole blood, serum, semen, saliva, tears, urine, fecal material, sweat, buccal smears, skin, and biopsies of specific organ tissues.

8. The method of claim 1, wherein bone erosion is used as a further biomarker.

9. The method of claim 1, wherein the amounts of the biomarkers are determined by means of a reagent which specifically binds to the biomarker proteins.

10. The method of claim 9, wherein the reagent is selected from the group consisting of an antibody, an antibody derivative and an antibody fragment.

11. The method of claim 9, wherein the determining of the amounts of the biomarkers includes at least one direct or indirect immunoassay selected from the group consisting of a competitive binding assay, a non-competitive binding assay, a radioimmunoassay, immunohistochemistry, an enzyme-linked immunosorbent assay (ELISA), a sandwich assay, a gel diffusion immunodiffusion assay, an agglutination assay, dot blotting, a fluorescent immunoassay such as fluorescence-activated cell sorting (FACS), a chemiluminescence immunoassay, an immunoPCT immunoassay, a protein A or protein G immunoassay, and an immunoelectrophoresis assay.

12. A composition for the prediction, diagnosis or prognosis of rheumatoid arthritis (RA) comprising specific detection agents for determining the amounts of the biomarkers consisting of TNF-alpha, MCP-1, IL-1ra, IL-6 and RF.

13. The method of claim 12, wherein the means specifically binding to a biomarker comprise at least one sequence-specific oligonucleotide which is able to bind specifically to the mRNAs encoding said biomarker.

14. The composition of claim 12, wherein the means specifically binding to a biomarker is selected from the group consisting of an antibody, an antibody derivative and an antibody fragment.

15. The composition of claim 14 wherein the means to comprise a monoclonal antibody.

16. The composition of claim 14, wherein the means specifically binding to a biomarker are immobilized on a solid support.

17-73. (canceled)
